(19) United States
(12) Patent Application Publication
(10) Pub. No.: US 2015/0327841 A1
(43) Pub. Date: Nov. 19, 2015

(54) TRACKING IN ULTRASOUND FOR IMAGING AND USER INTERFACE

(71) Applicants: KABUSHIKI KAISHA TOSHIBA, Tokyo (JP); Toshiba Medical Systems Corporation, Tochigi (JP)

(72) Inventors: Zoran BANJANIN, Bellevue, WA (US); Gilles D. GUENETTE, Sammamish, WA (US); Christopher J. SANDERS, Redmond, WA (US); Raymond F. WOODS, North Bend, WA (US)

(73) Assignees: KABUSHIKI KAISHA TOSHIBA, Tokyo (JP); Toshiba Medical Systems Corporation, Tochigi (JP)

(21) Appl. No.: 14/276,824

(22) Filed: May 13, 2014

(51) Int. Cl. A61B 8/08

(52) U.S. Cl. CPC A61B 8/5276

(57) ABSTRACT

An ultrasound probe and corresponding method of obtaining motion related data for the ultrasound probe. The probe including a housing, a transducer located inside said housing having transducer elements in a predetermined configuration that generates ultrasound, transmits the generated ultrasound towards an object, and receives echoes that have been reflected from the object, a motion sensing device having a fixed position with respect to said housing and including a sensor configured to generate a movement signal indicative of at least one type of movement of the ultrasound probe, and processing circuitry configured to generate images from the echoes received by the transducer and to correct the generated images by image correlation based on the generated movement signal.

### FIELD

Embodiments described herein relate generally to ultrasound diagnostic imaging systems for and method of tracking a probe position using motion sensors for ultrasound diagnostic imaging systems.

### BACKGROUND

In the field of ultrasound medical examination, there have been some attempts to improve a user interface between the ultrasound imaging system and the operator. In general, an operator of an ultrasound scanner holds a probe to place it on a patient in an area of interest for scanning an image.

The probe position is tracked for certain purposes of the ultrasound imaging system. One exemplary purpose is to spatially register 2D and or 3D images with respect to the relative probe position. Spatially registered images are previously scanned or live images using the ultrasound imaging system are fused with other modality images or volumes such as computer tomography (CT) and magnetic resonance imaging (MRI). The fused images may be diagnostically useful in follow-ups for monitoring disease and or treatment progress.

One prior-art attempt provided a plurality of magnetic sensors for registering 2D ultrasound images with a probe position. Magnetic tracking has achieved fair market integration, with integrated magnetic sensors being incorporated into ultrasound systems. However, since these systems are somewhat complicated to setup and have a high cost, they have not been widely accepted by users and are mainly used for multimodal image fusion.

In the last few years, microelectromechanical (“MEM”) based gyroscopes and accelerometers have been widely introduced in various high volume consumer devices, including smartphones, tablets, gaming devices, remote controllers, etc. Motion sensor MEMs are highly integrated and are able to provide 9-axis motion sensing with a 3-axis accelerometer, a 3-axis gyroscope and a 3-axis magnetic compass. Due to high volume manufacturing, motion sensors have a relatively low cost and developments in the quality and performance of these sensors are increasing rapidly.

Another prior-art attempt provided an optical system of image registration. The optical system included stereo optical cameras on a tall stand and a large target probe attachment. These additional pieces of the equipment are not practical for use with the ultrasound imaging system due to their size and costs.

In view of the above described exemplary prior-art attempts, the field of ultrasound imaging still needs an improved method and device for tracking a probe position during the examination sessions.

### BRIEF DESCRIPTION OF THE DRAWINGS

A more complete appreciation of the embodiments described herein, and many of the attendant advantages thereof will be readily obtained as the same becomes better understood by reference to the following detailed description when considered in connection with the accompanying drawings, wherein:

FIG. 1 is a schematic diagram illustrating an embodiment of the ultrasound diagnosis apparatus;

FIG. 2A is a diagram illustrating a first embodiment of the probe tracking device in the ultrasound diagnosis apparatus;

FIG. 2B is a diagram illustrating a second embodiment of the probe tracking device in the ultrasound diagnosis apparatus;

FIG. 2C is a diagram illustrating a third embodiment of the probe tracking device in the ultrasound diagnosis apparatus;

FIG. 3A is a diagram illustrating a first embodiment of the probe tracking device, which is mounted on a top of a display unit;

FIG. 3B is a diagram illustrating a second embodiment of the probe tracking device, which is integrated in a top of a display unit;

FIG. 4 is a diagram illustrating an exemplary operation of one embodiment of the probe tracking device in the ultrasound imaging and diagnosis system;

FIG. 5 is a diagram illustrating an exemplary operation of another embodiment of the probe tracking device in the ultrasound imaging and diagnosis system;

FIG. 6 is a flow chart illustrating steps involved in one process of tracking a probe in the ultrasound imaging and diagnosis system;

FIG. 7 is a diagram illustrating steps involved in one process of tracking a probe position and utilizing the position information in the ultrasound imaging and diagnosis system;

FIG. 8 is a diagram illustrating an exemplary display of tracking a combination of a probe and a patient in the ultrasound imaging system;

FIG. 9 is a diagram illustrating a 3D image display as an exemplary application of the operator positional tracking in the image display system;

FIG. 10 is a schematic diagram illustrating an embodiment of the ultrasound diagnosis apparatus;

FIG. 11A is a handheld probe having a motion sensor embedded therein and a slide stopper according to an embodiment;

FIG. 11B is an example of a wobbler probe system;

FIG. 12A is a handheld probe having a motion sensor embedded therein according to an embodiment;

FIG. 12B is an example of a probe in slider ABUS system;

FIG. 13A is an ultrasound device having a motion sensor embedded therein;

FIG. 13B is an example of a warm bath ultrasound ABUS-alternative system;

FIG. 14 is a schematic diagram illustrating an embodiment of the ultrasound diagnosis apparatus;

FIG. 15 is a schematic diagram illustrating an embodiment of the ultrasound diagnosis apparatus;

FIG. 16 is a diagram illustrating an exemplary operation of an embodiment of the probe operated interface in the ultrasound imaging and diagnosis system;

FIG. 17 is a diagram illustrating a motion sensor based tracking device according to an embodiment;

FIG. 18 is a diagram illustrating a motion sensor based tracking device together with an image tracking device according to an embodiment;

FIG. 19 is a flow diagram according to an embodiment; and

FIG. 20 is a schematic diagram illustrating a computing system according to an embodiment.

### DETAILED DESCRIPTION

According to one embodiment, an ultrasound probe includes a housing, a transducer located inside said housing having transducer elements in a predetermined configuration that generates ultrasound, transmits the generated ultrasound towards an object, and receives echoes that have been reflected from the object, a motion sensing device having a fixed position with respect to said housing and including a sensor configured to generate a movement signal indicative of at least one type of movement of the ultrasound probe and processing circuitry configured to generate images from the echoes received by the transducer and to correct the generated images by image correlation based on the generated movement signal.

According to another embodiment of the ultrasound probe, said motion sensing device is located inside said housing.

According to another embodiment of the ultrasound probe, said motion sensing device is located outside said housing.

According to another embodiment of the ultrasound probe, the ultrasound probe further includes a mounting frame for detachably mounting said motion sensing device on said housing.

According to another embodiment of the ultrasound probe, said motion sensing device includes any combination of a gyroscope, an accelerometer, and a compass.

According to another embodiment of the ultrasound probe, said motion sensing device includes at least a microelectromechanical (MEM) device.

According to another embodiment of the ultrasound probe, said motion sensing device generates the movement signal indicative any combination of a linear movement, a rotational movement, an angular movement, and acceleration.

According to another embodiment of the ultrasound probe, the ultrasound probe further includes a controller connected to said motion sensing device configured to selectively activate said motion sensing device.

According to another embodiment of the ultrasound probe, said processing circuitry is further configured to provide a warning to the user of the ultrasound probe when the movement signal indicates movement having a value greater than a predetermined threshold.

According to one embodiment there is included a method of obtaining motion related data for an ultrasound probe. The method includes the steps of providing a motion sensing device at a fixed position with respect to a housing of the ultrasound probe, generating a movement signal indicative of at least one type of movement of the ultrasound probe using a sensor of the motion sensing device, receiving echoes from an object that has been transmitted with ultrasound, generating images from the echoes received by the transducer, and correcting the generated images by image correlation based on the generated movement signal.

According to another embodiment of the method, the correcting further includes correlating images by compensating for linear movement in non-designated directions and rotational movement in non-designated axes.

According to another embodiment of the method, said providing step places the mot ion sensing device inside said housing.

According to another embodiment of the method, said providing step places the motion sensing device outside said housing.

According to another embodiment of the method, the method includes an additional step of detachably mounting the motion sensing device on the housing.

According to another embodiment of the method, the signal is generated using any combination of a gyroscope, an accelerometer, and a compass.

According to another embodiment of the method, said providing step uses at least a microelectromechanical (MEM) device.

According to another embodiment of the method, said generating step generates the signal indicative any combination of a linear movement, a rotational movement, an angular movement, and acceleration.

According to another embodiment of the method, the method includes an additional step of selectively activating the motion sensing device.

According to another embodiment of the method, the signal is generated while the probe is reciprocated over a surface of a patient in a substantially linear manner.

According to another embodiment of the method, the signal is generated while the probe is repeatedly wobbled at a pivoted area of a patient.

According to another embodiment of the method, the signal is generated as the probe is moved in a predetermined unique manner to indicate a predefined meaning during an ultrasound examination.

According to another embodiment of the method, the predefined meaning is a beginning of data collection.

According to another embodiment of the method, the predefined meaning is an ending of data collection.

According to another embodiment of the method, the method includes providing a warning to the user of the ultrasound probe when the movement signal indicates movement having a value greater than a predetermined threshold.

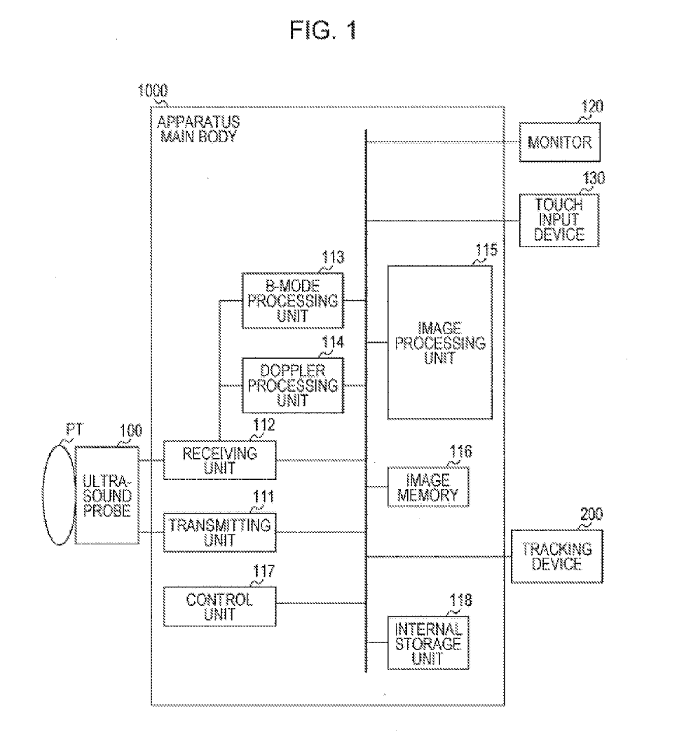

Exemplary embodiments of an ultrasound diagnosis apparatus will be explained below in detail with reference to the accompanying drawings. Like reference numerals designate identical or corresponding parts throughout the several views. Now referring to FIG. 1, a schematic diagram illustrates an embodiment of the ultrasound diagnosis apparatus.

The embodiment includes an ultrasound probe 100, a monitor 120, a touch input device 130, a tracking device 200 and an apparatus main body 1000. One embodiment of the ultrasound probe 100 includes a plurality of piezoelectric vibrators, and the piezoelectric vibrators generate ultrasound based on a driving signal supplied from a transmitting unit 111 housed in the apparatus main body 1000. The ultrasound probe 100 also receives a reflected wave from a subject Pt and converts the wave into an electric signal. Moreover, the ultrasound probe 100 includes a matching layer provided to the piezoelectric vibrators and a backing material that prevents propagation of ultrasound backward from the piezoelectric vibrators.

As ultrasound is transmitted from the ultrasound probe 100 to the subject Pt, the transmitted ultrasound is consecutively reflected by discontinuity planes of acoustic impedance in internal body tissue of the subject Pt and is also received as a reflected wave signal by the piezoelectric vibrators of the ultrasound probe 100. The amplitude of the received reflected wave signal depends on a difference in the acoustic impedance of the discontinuity planes that reflect the ultrasound. For example, when a transmitted ultrasound pulse is reflected by a moving blood flow or a surface of a heart wall, a reflected wave signal is affected by a frequency deviation. That is, due to the Doppler Effect, the reflected wave signal is dependent on a velocity component in the ultrasound transmitting direction of a moving object.

The apparatus main body 1000 ultimately generates signals representing an ultrasound image. The apparatus main body 1000 controls the transmission of ultrasound from the probe 100 towards a region of interest in a patient as well as the reception of a reflected wave at the ultrasound probe 100. The apparatus main body 1000 includes a transmitting unit 111, a receiving unit 112, a B-mode processing unit 113 implemented by processing circuitry, a Doppler processing unit 114 implemented by processing circuitry, an image processing unit 115 implemented by processing circuitry, an image memory 116, a control unit 117 implemented by processing circuitry and an internal storage unit 118, all of which are connected via internal bus. The apparatus main body 1000 also optionally includes a color processing unit implemented by processing circuitry.

The transmitting unit 111 includes a trigger generating circuit, a delay circuit, a pulsar circuit and the like and supplies a driving signal to the ultrasound probe 100. The pulsar circuit repeatedly generates a rate pulse for forming transmission ultrasound at a certain rate frequency. The delay circuit controls a delay time in a rate pulse from the pulsar circuit for utilizing each of the piezoelectric vibrators so as to converge ultrasound from the ultrasound probe 100 into a beam and to determine transmission directivity. The trigger generating circuit applies a driving signal (driving pulse) to the ultrasound probe 100 based on the rate pulse.

The receiving unit 112 includes an amplifier circuit, an analog-to-digital (A/D) converter, an adder and the like and creates reflected wave data by performing various processing on a reflected wave signal that has been received at the ultrasound probe 100. The amplifier circuit performs gain correction by amplifying the reflected wave signal. The A/D converter converts the gain-corrected reflected wave signal from the analog format to the digital format and provides a delay time that is required for determining reception directivity. The adder creates reflected wave data by adding the digitally converted reflected wave signals from the A/D converter. Through the addition processing, the adder emphasizes a reflection component from a direction in accordance with the reception directivity of the reflected wave signal. In the above described manner, the transmitting unit 111 and the receiving unit 112 respectively control transmission directivity during ultrasound transmission and reception directivity during ultrasound reception.

The apparatus main body 1000 further includes the B-mode processing unit 113 and the Doppler processing unit 114, which are each implemented by processing circuitry. The B-mode processing unit 113 receives the reflected wave data from the receiving unit 112, performs logarithmic amplification and envelopes detection processing and the like so as to create B-mode data for representing a signal strength by the brightness. The Doppler processing unit 114 performs frequency analysis on velocity information from the reflected wave data that has been received from the receiving unit 112. The Doppler processing unit 114 extracts components of a blood flow, tissue and contrast media echo by Doppler effects. The Doppler processing unit 114 generates Doppler data on moving object information such as an average velocity, a distribution, power and the like with respect to multiple points.

The apparatus main body 1000 further includes additional units implemented by processing circuitry that are related to image processing of the ultrasound image data. The image processing unit 115 generates an ultrasound image from the B-mode data from the B-mode processing unit 113 or the Doppler data from the Doppler processing unit 114. Specifically, the image processing unit 115 respectively generates a B-mode image from the B-mode data and a Doppler image from the Doppler data. Moreover, the image processing unit 115 converts or scan-converts a scanning-line signal sequence of an ultrasound scan into a predetermined video format such as a television format. The image processing unit 115 ultimately generates an ultrasound display image such as a B-mode image or a Doppler image for a display device. The image memory 116 stores ultrasound image data generated by the image processing unit 115.

The control unit 117 implemented by processing circuitry controls overall processes in the ultrasound diagnosis apparatus. Specifically, the control unit 117 controls processing in the transmitting unit 111, the receiving unit 112, the B-mode processing unit 113, the Doppler processing unit 114 and the image processing unit 115 based on various setting requests that are inputted by the operator via the input devices and control programs and setting information that are read from the internal storage unit 118. For Example, the control programs executes certain programmed sequence of instructions for transmitting and receiving ultrasound, processing image data and displaying the image data. The setting information includes diagnosis information such as a patient ID and a doctor's opinion, a diagnosis protocol and other information. Moreover, the internal storage unit 118 is optionally used for storing images stored in the image memory 116. Certain data stored in the internal storage unit 118 is optionally transferred to an external peripheral device via an interface circuit. Lastly, the control unit 117 also controls the monitor 120 for displaying an ultrasound image that has been stored in the image memory 116.

A plurality of input devices exist in embodiments of the ultrasound diagnosis apparatus. Although the monitor or display unit 120 generally displays an ultrasound image as described above, a certain embodiment of the display unit 120 additionally functions as an input device such as a touch panel alone or in combination with other input devices for a system user interface for the first embodiment of the ultrasound diagnosis apparatus. The display unit 120 provides a Graphical User Interface (GUI) for an operator of the ultrasound diagnosis apparatus to input various setting requests in combination with the input device 130. The input device 130 includes a mouse, a keyboard, a button, a panel switch, a touch command screen, a foot switch, a trackball, and the like. A combination of the display unit 120 and the input device 130 optionally receives predetermined setting requests and operational commands from an operator of the ultrasound diagnosis apparatus. The combination of the display unit 120 and the input device 130 in turn generates a signal or instruction for each of the received setting requests and or commands to be sent to the apparatus main body 1000. For example, a request is made using a mouse and the monitor to set a region of interest during an upcoming scanning session. Another example is that the operator specifies via a processing execution switch a start and an end of image processing to be performed on the image by the image processing unit 115.

Still referring to FIG. 1, a plurality of input devices in the embodiment of the ultrasound diagnosis apparatus additionally includes an external tracking device 200. One embodiment of the tracking device 200 is connected to the apparatus main body 1000 via predetermined wired or wireless connection for sending position data or information of the probe 100 in the ultrasound diagnosis apparatus. For example, the probe position data includes at least a predetermined set of absolute or relative positional information of the probe 100 with respect to or within a predetermined area or space. However, the probe position data is not limited to positional data and optionally includes other information such as the angle of the probe with respect to a predetermined coordinate. Furthermore, the tracking device 200 obtains the positional information of any combination of the probe 100, a patient and an operator with respect to or within a predetermined area or space.

One embodiment of the tracking device 200 includes other devices such as a space measuring device for measuring at least distance and angle of the probe based upon emitted electromagnetic radiation that is emitted towards the probe and reflected electromagnetic radiation that is reflected from the probe and a processing device connected to the space measuring device for determining a change in distance and angle of the probe in space based upon on the emitted electromagnetic radiation and the reflected electromagnetic radiation.

Another embodiment of the tracking device 200 includes any combination of infrared (IR) depth sensors, optical cameras, accelerometers, gyroscopes and microphones for identifying and locating any combination of a probe, an operator and a patient in a predetermined space with respect to the ultrasound diagnosis apparatus according to the current invention. For example, the microphone is utilized to identify a patient and or an operator based upon the voice analysis. The microphone may be also utilized to determine an approximate direction of a patient and or an operator with respect to the location of microphone based upon the voice analysis. Another example is that a combination of an accelerometer and a gyroscope is optionally mounted on a probe to determine an amount of change in movement, angle and or direction. Other exemplary sensors such as an IR depth sensor and an optical camera are optionally used to detect an amount of movement of a predetermined object such as a probe, an operator and a patient.

Another embodiment of the tracking device 200 corresponds to the tracking device being incorporated into the probe 100. For instance, the tracking device 200 may include the incorporation of microelectromechanical (“MEM”) based motion sensor MEMs into the probe 100. These motion sensors provide 9-axis motion sensing with a 3-axis accelerometer, a 3-axis gyroscope and a 3-axis magnetic compass which sense a large amount of information about the position and movement of the probe 100.

In embodiments of the ultrasound diagnosis apparatus, the tracking device 200 is not necessarily limited to perform the above described functions in an exclusive manner. In other embodiments of the ultrasound diagnosis apparatus, the tracking device 200 performs together with other devices such as the image processing unit 115 and the control unit 117 or movement processing unit to accomplish the above described functions including the determination of the positional and angular data of the probe 100.

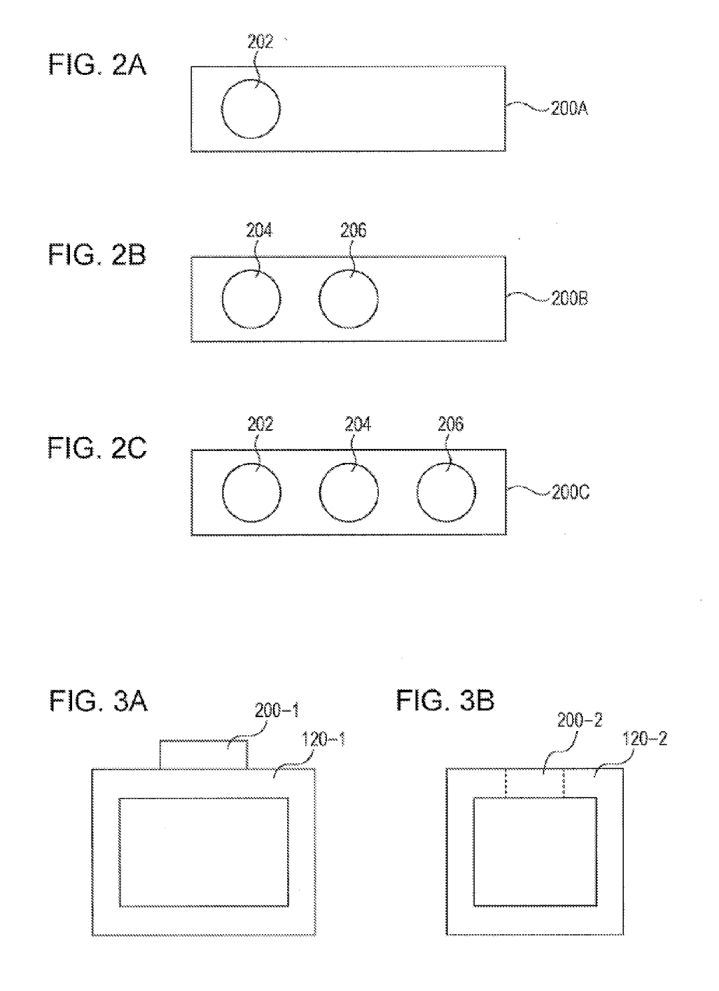

Now referring to FIG. 2A, a diagram illustrates one embodiment of a tracking device 200A in the ultrasound diagnosis apparatus according to the current invention. The tracking device 200A generally includes a camera or an image optical sensor 202 for capturing an image of the probe 100. The tracking device 200A optionally includes other units such as auto focus unit, a light source and so on. The above sensors in the tracking device 200A alone or in combination with other sensors detect a shape, a depth and or a movement of the probe 100 so as to generate a predetermined set of information or data such as positional data and angular data. The above sensors are merely illustrative, and the tracking device 200A according to the current invention is not limited to a particular set of sensors or sensing modes for detecting the probe 100. To facilitate the detection, the probe 100 is optionally marked or colored in predetermined manner so that the probe 100 is visibly enhanced.

Now referring to FIG. 2B, a diagram illustrates another embodiment of the tracking device 200B in the ultrasound diagnosis apparatus according to the current invention. The tracking device 200B includes an infrared (IR) light source 204 and certain sensors such as an IR light sensor 206. The infrared (IR) light source 204 emits infrared towards the probe 100 wile the IR light sensor 206 receives the infrared reflected from the probe 100. Although the illustrated embodiment of the tracking device 200B has a separate position for the IR light source 204 and the IR light sensor 206, the position may be identical. Furthermore, the infrared range is not also limited and is optionally outside of the IR range of the electromagnetic radiation. The above sensors in the tracking device 200B alone or in combination with other sensors detect a shape, a depth and or a movement of the probe 100 so as to generate a predetermined set of information or data such as positional data and angular data. The above sensors are merely illustrative, and the tracking device 200B according to the current invention is not limited to a particular set of sensors or sensing modes for detecting the probe 100.

Now referring to FIG. 2C, a diagram illustrates yet another embodiment of the tracking device 200C in the ultrasound diagnosis apparatus according to the current invention. The tracking device 200C includes a camera or an image optical sensor 202 for capturing an image of the probe 100. The tracking device 200C also includes an infrared (IR) light source 204 and certain sensors such as an IR light sensor 206. The infrared (IR) light source 204 emits infrared towards the probe 100 while the IR light sensor 206 receives the infrared reflected from the probe 100. Although the illustrated embodiment of the tracking device 200C has a separate position for the IR light source 204 and the IR light sensor 206, the position may be identical. Furthermore, the infrared range is not also limited and is optionally outside of the IR range of the electromagnetic radiation. The above multiple sensors in the tracking device 200C alone or in combination with other sensors detect a shape, a depth and or a movement of the probe 100 so as to generate a predetermined set of information or data such as positional data and angular data. In the above exemplary embodiment, the tracking device 200C emits and receives invisible electromagnetic radiation while it also captures an image using the visible light range. The above sensors are merely illustrative, and the tracking device 200C according to the current invention is not limited to a particular set of sensors or sensing modes for detecting the probe 100.

The above described embodiments are merely illustrative of the inventive concept of tracking a probe in the ultrasound diagnostic imaging system. In general, the larger a number of the above described sensors the probe tracking device has, the more accurate the predetermined information the probe tracking device generates, assuming that the resolution of the sensors is not yet met by a plurality of the sensors. Furthermore, the accuracy of the information depends upon the resolution of the sensors.

Now referring to FIGS. 3A and 3B, the tracking device 200 is implemented in various manners in the ultrasound diagnosis apparatus. FIG. 3A illustrates an embodiment of a tracking device 200-1, which is mounted on a top of a display unit 120-1. The mounting is not limited on the top of the display unit 120-1 and includes any other surfaces of the display unit 120-1 or even other units or devices in or outside the ultrasound diagnosis apparatus according to the current invention. Depending upon implementation, the tracking device 200-1 is optionally mounted on the display unit 120-1 in a retrofitted manner in an existing ultrasound diagnosis apparatus system. One embodiment of the tracking device 200-1 includes an IR light and a depth image detector.

FIG. 3B illustrates a second embodiment of a tracking device 200-2, which is integrated in a top portion of a display unit 120-2 as indicated by the dotted lines. The integration is not limited to the top portion of the display unit 120-2 and includes any other portions of the display unit 120-2 or even other units or devices in the ultrasound diagnosis apparatus according to the current invention. One embodiment of the tracking device 200-2 includes an IR light and a depth image detector.

As already described with respect to FIGS. 2A, 2B and 2C, one embodiment of the probe tracking device is a separate unit and is placed next to a predetermined location near an existing device such as a display unit. The placement is not limited to the side of the display unit and includes any other locations or even other units or devices in or outside the ultrasound diagnosis apparatus according to the current invention. Depending upon implementation, the probe tracking device is optionally placed near the display unit or other devices to be incorporated into in an existing ultrasound diagnosis apparatus system in a retrofitted manner.

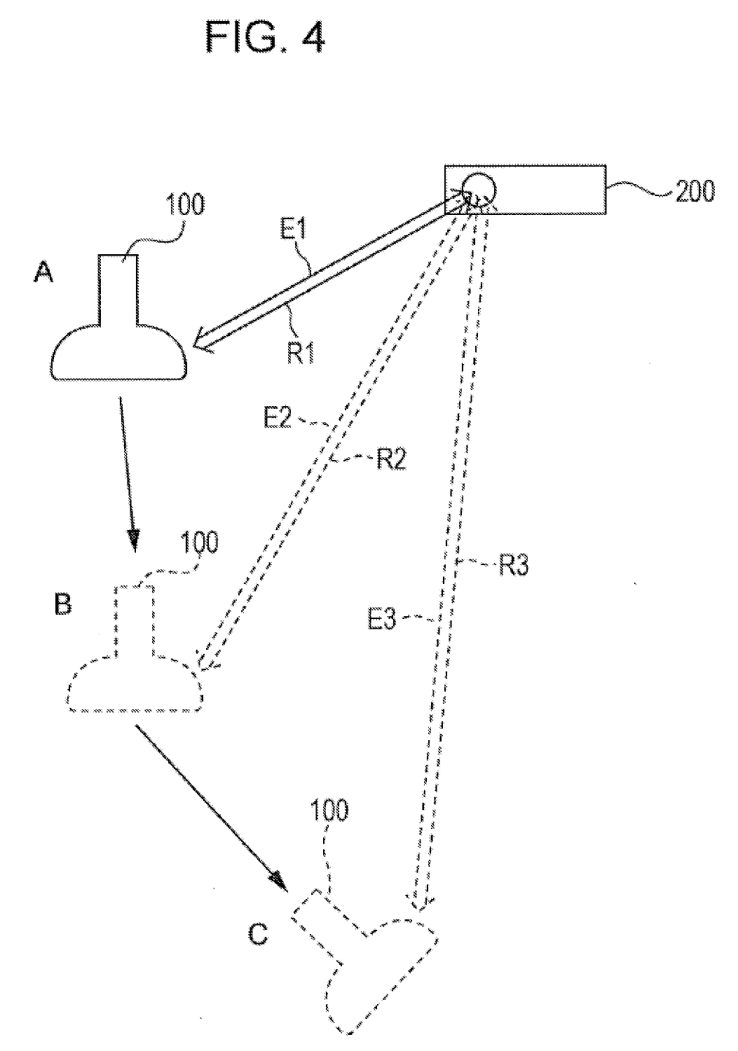

Now referring to FIG. 4, a diagram illustrates an exemplary operation of one embodiment of the probe tracking device in the ultrasound imaging and diagnosis system. For example, while an operator OP holds the probe 100 for scanning an ultrasound image, the tracking device 200 emits a predetermined range of electromagnetic radiation or light towards the probe 100 as indicated as E1 at a position A. Although only a single ray E1 is illustrated, the tracking device 200 generally emits a group of rays in certain broad directions from a predetermined stationary position. The predetermined rage of the electromagnetic radiation includes both visible and invisible range and is not limited to a particular narrow range. As the electromagnetic radiation that has been emitted from the tracking device 200 reaches the probe 100, the emitted electromagnetic radiation is reflected on a surface of the probe 100.

The probe 100 reflects the emitted light back towards the tracking device 200 as indicated as R1 from the position A. The tracking device 200 receives the reflected electromagnetic radiation. The tracking device 200 determines a change in distance and angle of the probe 100 in a predetermined space based upon on the emitted electromagnetic radiation and the reflected electromagnetic radiation. Lastly, the tracking device 200 outputs the change in an ultrasound imaging system. In one example, a display unit displays the change. In another example, the ultrasound imaging system uses the change in the probe position for a particular application such as stitching the previously stored images as will be further described.

In this example, it is assumed that the probe 100 is not stationary as indicated by the arrows and dotted lines. That is, the probe 100 moves from the position A to a position C via a position B. As the probe 100 moves from one position to another, the tracking device 200 continuously monitors the position and the angle of the probe 100 by repeatedly emitting the predetermined range of electromagnetic radiation towards the probe 100 and receiving the reflected electromagnetic radiation from the probe 100. At the position B, the tracking device 200 respectively emits and receives the electromagnetic radiation rays E2 and R2 as indicated in dotted lines to and from the probe 100. By the same token, at the position C, the tracking device 200 respectively emits and receives the electromagnetic radiation rays E3 and R3 as indicated in dotted lines to and from the probe 100. As the tracking device 200 monitors the moving probe 100, the tracking device 200 determines a change in distance and angle of the probe 100 in a predetermined space based upon on the emitted electromagnetic radiation rays E1, E2, E3 and the reflected electromagnetic radiation rays R1, R2, R3.

In order to have an efficient and accurate monitoring operation, the electromagnetic radiation is reflected from the probe. Although the reflecting surface of the probe 100 is not limited to any particular surface, one embodiment of the probe 100 is optionally manufactured to have a predetermined coat that is suitable for reflecting a particular frequency range of light. In another embodiment, the probe 100 is optionally manufactured to have a predetermined reflector element in lieu of the coating surface.

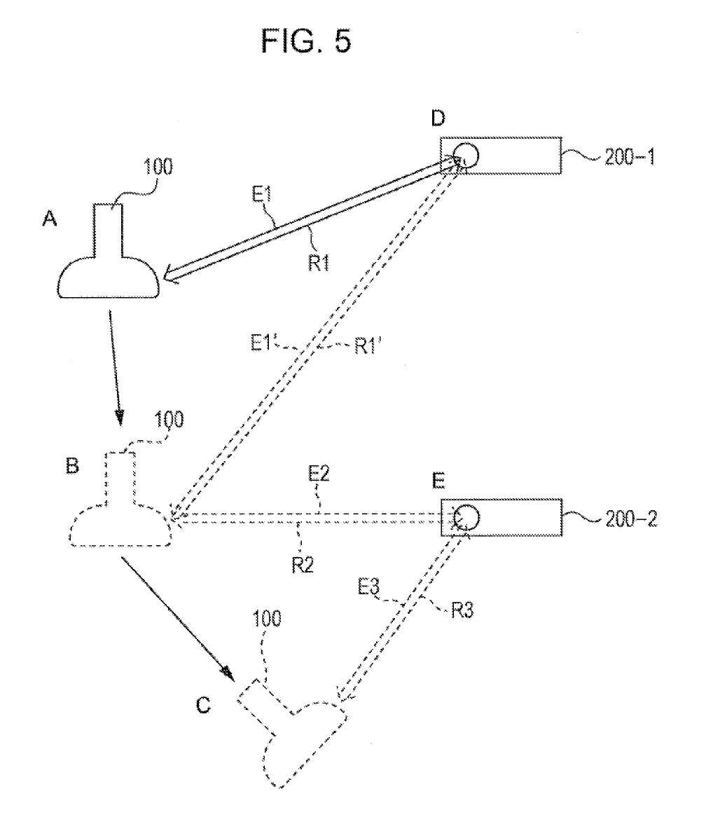

Now referring to FIG. 5, a diagram illustrates an exemplary operation of one embodiment of the probe tracking device in the ultrasound imaging and diagnosis system. For example, while an operator OP holds a probe 100 for scanning an ultrasound image, a tracking device 200-1 emits a predetermined range of electromagnetic radiation or light towards the probe 100 as indicated as E1 at a position A. Although only a single ray E1 is illustrated, the tracking device 200-1 generally emits a plurality of rays in certain broad directions. The predetermined rage of the electromagnetic radiation includes both visible and invisible range and is not limited to a particular range.

In the above exemplary embodiment, it is assumed that the probe 100 is not stationary as indicated by the arrows and dotted lines. That is, the probe 100 moves from the position A to a position C via a position B. As the probe 100 moves from one position to another, the tracking device 200-1 continuously monitors the position and the angle of the probe 100 by repeatedly emitting the predetermined range of electromagnetic radiation towards the probe 100 and receiving the reflected electromagnetic radiation from the probe 100. At the same time, a second tracking device 200-2 also continuously monitors the position and the angle of the probe 100 by repeatedly emitting the predetermined range of electromagnetic radiation towards the probe 100 and receiving the reflected electromagnetic radiation from the probe 100. The tracking device 200-1 is located at a position D while the tracking device 200-2 is located at a position E through-out the course of monitoring the probe 100.

In the above exemplary embodiment of the ultrasound imaging and diagnosis system, a plurality of the probe tracking devices simultaneously monitor the positional and or angular change of the probe 100 in a predetermined space. That is, when the probe 100 is at the position A, the tracking device 200-1 at the position D alone emits and receives respective electromagnetic radiation rays E1 and R1 as indicated in dotted lines to and from the probe 100. When the probe 100 is at the position B, the probe tracking devices 200-1 and 200-2 both emit respective electromagnetic radiation rays E1′ and E2. When the probe 100 is also at the position B, the probe tracking devices 200-1 and 200-2 respectively receive the electromagnetic radiation rays R1′ and R2. On the other hand, when the probe 100 is at the position C, the tracking device 200-2 at the position E alone emits and receives respective electromagnetic radiation rays E3 and R3 as indicated in dotted lines to and from the probe 100.

Still referring to FIG. 5, as the probe tracking devices 200-1 and 200-2 monitor the moving probe 100, the probe tracking devices 200-1 and 200-2 in combination determine a change in distance and angle of the probe 100 in a predetermined space based upon on the emitted electromagnetic radiation rays E1, E1′, E2, E3 and the reflected electromagnetic radiation rays R1, R1′, R2, R3. In the above exemplary embodiment, it is assumed that the probe tracking devices 200-1 and 200-2 are located respectively at the positions D and E in a fixed manner. In another embodiment, any combination of the probe 100 and the probe tracking devices 200-1 and 200-2 is optionally moving during the course of monitoring the position and or the angle of the probe 100 in a predetermined space. Furthermore, the movement of the probe 100, the tracking device 200-1 or the tracking device 200-2 is not necessarily coordinated or synchronous.

In alternative embodiment, a single probe tracking device houses a plurality of spatially separated sensors to monitor the moving probe 100 and to determine a change in distance and angle of the probe 100 in a predetermined space based upon on the electromagnetic radiation rays.

With respect to FIGS. 4 and 5, the use of electromagnetic radiation is not limited to a particular range and includes at least infrared radiation and or visible radiation. Although the diagrams in FIGS. 4 and 5 do not explicitly illustrate, the use of electromagnetic radiation requires a plurality of hardware and software for sensing movement and angle. When visible light is used, one embodiment of the tracking device 200 includes a predetermined sensor such as a stereoscopic optical sensor to estimate depth dimension based upon images that have been captured by at least two spatially separated cameras. In case of visible light, electromagnetic radiation is not necessarily emitted from a particular source if a sufficient amount of visible Eight is available in the environment.

Still referring to FIGS. 4 and 5, in other embodiments of the tracking device 200, additional techniques are used. In one embodiment, infrared is used with a predetermined light coding technique to estimate depth dimension. The observed volume is coded by infrared, and a predetermined single CMOS depth image sensor detects the coded light from the observed volume. Furthermore, a “time-of-flight” technique is optionally used in another embodiment to acquire depth based upon a 3D camera or a time-of-flight camera for measuring the time-of-flight of a light signal between the camera and the subject for each point of the image. The time-of-flight camera is a class of scannerless Light Detection and Ranging (LIDAR) in which the entire image is captured with each laser or light pulse as opposed to point-by-point with a laser beam such as in scanning LIDAR systems. The light pulse includes ultraviolet, visible, or near infrared light. In order to practice the probe tracking, any combination of the above described techniques is implemented to determine the depth, movement and or angle of the probe within or with respect to a predetermined space in relation to the ultrasound imaging and diagnosis system.

FIGS. 4 and 5 illustrate that the tracking device 200 monitors and determine the movement of the probe 100 as an example. The tracking device 200 is not limited to track the movement of the probe 100 and is optionally used to monitor a plurality of predetermined objects in a simultaneous manner. In one embodiment, the tracking device 200 monitors the movement of any combination of the probe 100, a patient on which the probe is placed and an operator who places the probe 100 on the patient using a predetermined set of the sensors as described above. In this regard, one embodiment of the tracking device 200 provides multiple sets of relative or absolute positional and angular data for the predetermined objects in a continuous manner.

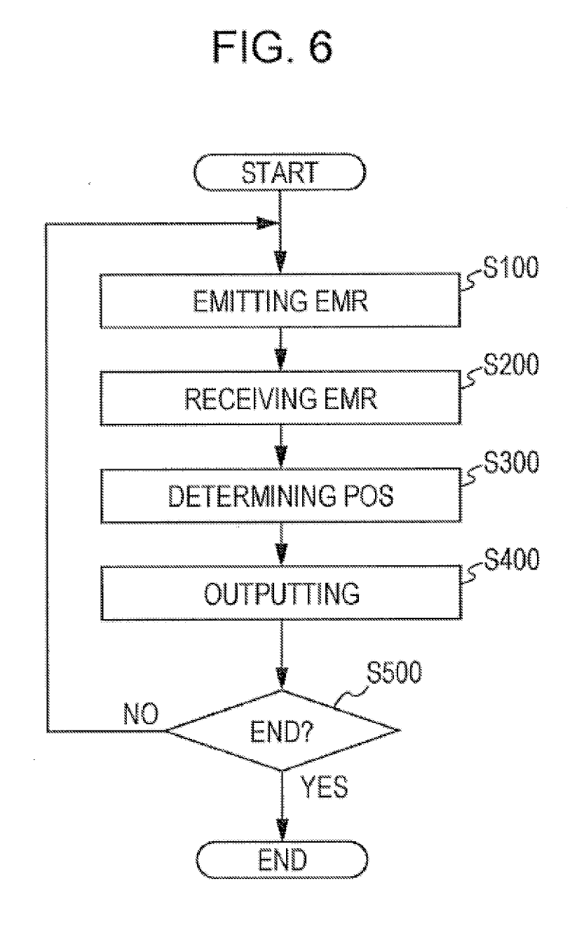

Now referring to FIG. 6, a flow chart illustrates steps involved in one process of tracking a probe in the ultrasound imaging and diagnosis system. The flow chart is exemplary and general and is not limited to a particular probe tracking process of the current invention. For these reasons, the electro-magnetic radiation (EMR) is utilized to include at least visible light range and an infrared range of the electromagnetic spectrum. On the other hand, the probe tracking process is not limited to using a particular range of the electromagnetic spectrum and or a particular combination of the sensors. In a step S100, a predetermined range or ranges of EMR is emitted from a predetermined position towards a probe to be tracked. If a visible range is utilized, it is not necessarily emitted from a particular source unless there is not a sufficient amount of visible light is available in a predetermined space where the probe is tracked. In this regard, the step S100 of emitting is optionally tantamount to providing EMR if visible light is available from the environment.

In a step S200, the EMR that has been substantially reflected from the probe is received in one embodiment of the current process. In another embodiment, while the EMR may be partially absorbed by the probe, EMR is still partially reflected from the probe and also received in the step S200. Thus, the predetermined range or range or ranges of EMR are received by a predetermined detector or sensor from the probe to be tracked. If a visible range is utilized, an image is captured by an optical camera. On the other hand, if a predetermined laser beam is used, a LIDAR camera captures the laser data. In any case, some reflected EMR is received in the step S200 at a predetermined position with respect to the emitting position of the step S100. In one embodiment of the current process, the received position and the emitting position are substantially identical. In another embodiment of the current process, the received position and the emitting position are substantially different. In this regard, there may be a substantial delay in emitting and receiving between the steps S100 and S200.

The steps S100 and S200 are performed in a variety of manners according to the current invention. For example, the emitting and receiving steps S100 and S200 are automatically activated and continuously performed only when the probe is in motion in one embodiment of the current process. In another embodiment of the current process, the steps S100 and S200 are not performed while the probe is stationary. In yet another embodiment of the current process, the steps S100 and S200 are manually activated to perform.

In a step S300, spatial information of the probe is determined according to the emitted EMR in the step S100, the received EMR in the step S200. In one embodiment of the current process, the emitted EMR in the step S100 is visible, and the received EMR in the step S200 is an image of the probe. The step S300 determines the spatial information of the probe based upon the images in the above visible EMR embodiment. On the other hand, in another embodiment of the current process, the emitted EMR in the step S100 is infrared, and the received EMR in the step S200 is infrared EMR data of the probe. The step S300 determines the spatial information of the probe based upon the infrared EMR data in the above infrared EMR embodiment. In yet another embodiment of the current invention, both the visible range and the infrared range of EMR are utilized, and the step S300 determines the spatial information of the probe based upon a combination of the images and the infrared EMR data. In any case, the spatial information includes any combination of absolute coordinates, relative movement in distance, speed, acceleration and angular change of the probe within the predetermined space.

After determining the spatial information in the step S300, the spatial information is outputted in a step S400 of the current process of tracking the probe in the ultrasound imaging and diagnosis system. In one embodiment of the current process, the outputting step S400 involves displaying of the data. For example, the displayed data is one of a 2D image, a 3D image and a 4D image that are based upon previously stored data and that corresponds to the change in spatial information with respect to the probe. Another exemplary displayed data is a 3D volume that is stitched together from a plurality of previously stored 3D volumes. Yet another exemplary displayed data is a 3D volume that is stitched together from a plurality of previously stored 2D images. An additional exemplary displayed image is an image that is based upon imaging data that is acquired by the probe that has been monitored for tracking.

Still referring to FIG. 6, the above described steps S100 through S400 are repeated until a predetermined condition is achieved in a step S500 in one embodiment of the current process. For example, the steps S100 through S400 are automatically activated and continuously performed while the probe is determined to be in motion in the step S500 in one embodiment of the current process. In another embodiment of the current process, the steps S100 through S400 are manually deactivated in the step S500.

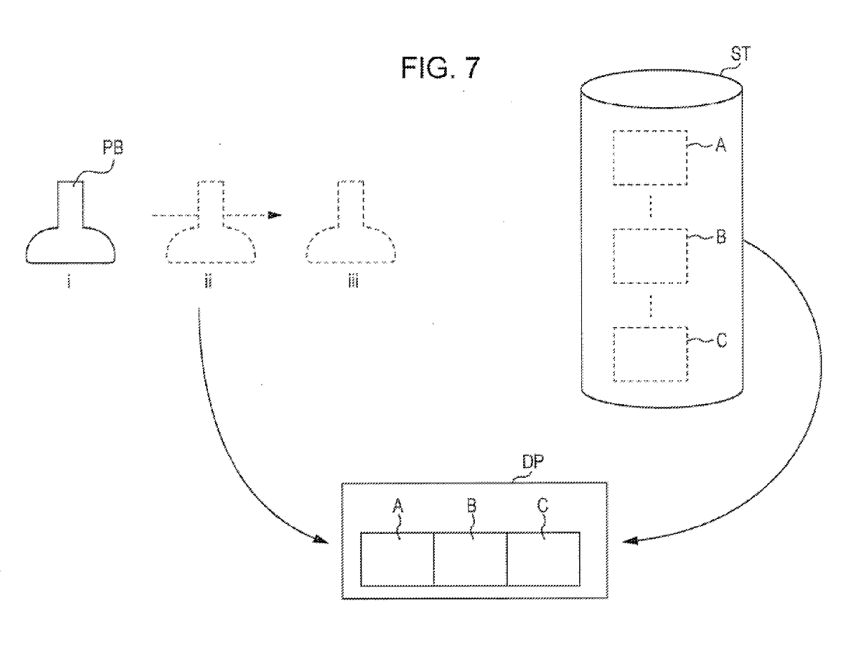

Now referring to FIG. 7, a diagram illustrates steps involved in one process of tracking a probe position and utilizing the position information in the ultrasound imaging and diagnosis system. In an exemplary process, a probe PB is moved from a first position i to a third position iii through a second position ii over a patient's body surface in order to scan a region of interest for ultrasound imaging. As the probe PB travels, the above described process as illustrated in the flow chart of FIG. 6 determines an amount of the probe movement in direction and or angle based upon the electromagnetic radiation as detected with respect to the probe PB. Alternatively, the process shown in FIG. 7 can be operated with movement and direction information or movement or direction information obtained from motion sensors.

Based upon the probe tracking information as determined by the above described process as illustrated in the flow chart of FIG. 6, a set of previously stored images are selected from a storage device ST. The previously stored images includes the region of interest that has been currently scanned by the probe PB and are generally acquired by an imaging and diagnosis system of modalities such as Xray-based computer tomography (CT) and magnetic resonance imaging (MRI), which generally provides a higher resolution than the ultrasound imaging. A corresponding set of the high-resolution images is selected from the storage device ST for displaying based upon the probe tracking information as indicated by the arrows. For example, as the probe PB travels from the first position i to the third position iii through the second position ii, the corresponding images A, B and C are optionally displayed on a monitor DP in a predetermined manner. The images A, B and C are sequentially displayed in a real time in one implementation mode while they may be stitched together in another implementation mode. The previously stored images are not limited to a different modality and may also optionally include ultrasound images.

Still referring to FIG. 7, the displayed data additionally include other images that are generated from a variety of previously stored images data. For example, the displayed image is one of a 2D image, a 3D image and a 4D image that are based upon previously stored data and that corresponds to the change in spatial information with respect to the probe. Another exemplary displayed image is a 3D volume that is stitched together from a plurality of previously stored 3D volumes. Yet another exemplary displayed data is a 3D volume that is stitched together from a plurality of previously stored 21) images. An additional exemplary displayed image is an image that is based upon imaging data that is acquired by the probe that has been monitored for tracking according to a process of the current invention.

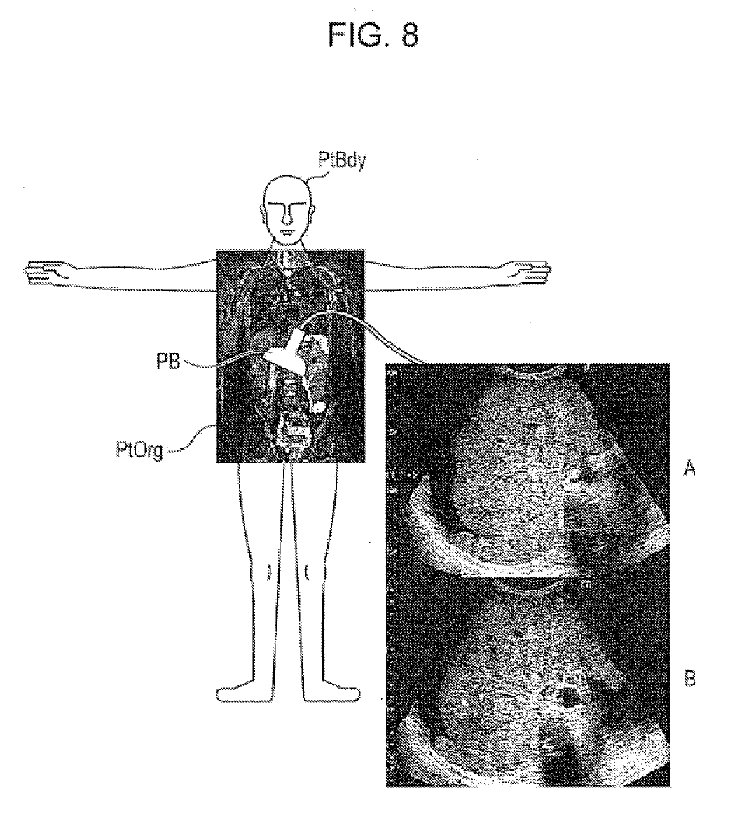

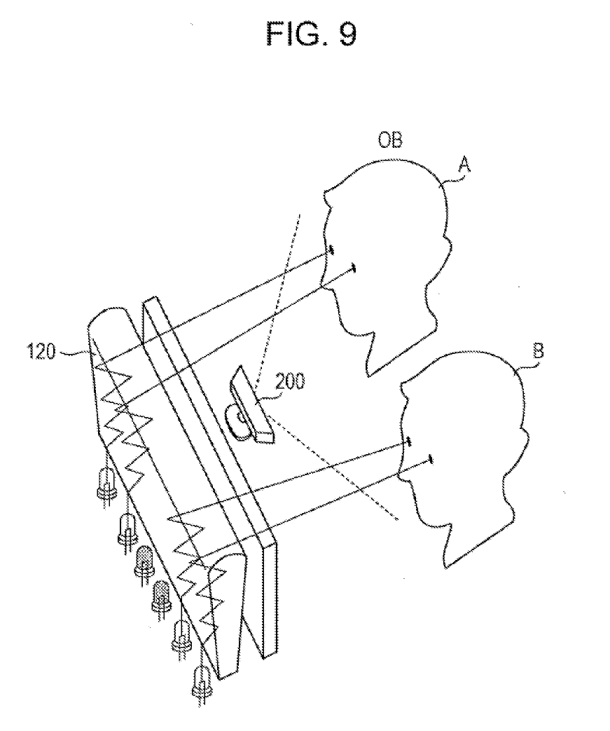

Now referring to FIG. 8, a diagram illustrates an exemplary display of tracking a combination of a probe and a patient in the ultrasound imaging system. In this exemplary display, a patient is lying down on his back, and the legs and arms are extended as shown in a patient image PtBdy. The patient image PtBdy is captured by a predetermined camera or 3D capturing device and stored. By the same token, a patient organ image PtOrg is previously captured by a conventional X-ray, magnetic resonance imaging (MRI) or computed tomography (CT) scanner. In one exemplary display, the patient organ image PtOrg is superimposed on the patient image PtBdy. Although the body image and the internal organ image are both extensive in the exemplary display, either or both of the images are optionally localized to a smaller portion of the body or the organ(s) for display. In a certain implementation, the above images, are optionally zoomed.

In an exemplary process, a probe PB is moved to a current probe position i on a patient's body surface in order to scan a region of interest for ultrasound imaging. The current position i of the probe PB is determined with respect to the patient body PtBdy, and an ultrasound image A is displayed at the current probe position i. As the current position i changes, the ultrasound image A also changes unless the operator optionally freezes the image A. After the operator determines a desirable ultrasound image for a particular organ of interest, the relevant positional information is stored along with the scanned ultrasound image at the established position I for the future use. Subsequently, the ultrasound image is scanned at the exact previously established probe position I for various purposes. For example, the chronologically scanned images are compared to determine the effect of a cancer treatment on the organ at the exactly identical location. Assuming that an ultrasound image B is previously scanned image before a predetermined treatment, the comparison of the images A and B are effective in determining the effect of the treatment.

Still referring to FIG. 8, to have an effective comparison in the above example, the ultrasound images A and B have to be scanned at the exactly identical location of the same organ. To facilitate the above identification task, as the operator moves the probe PB over the patient body PtBdy to identify the previously established probe position I with a visual aid yin the ultrasound imaging system. For example, a predetermined icon indicates the current probe position i on the image of the patient body PtBdy to provide a visual feedback to the operator who is trying to identify the previously established position I, which is also indicated by another predetermined icon. As the probe PB moves, the above described process as illustrated in the flow chart of FIG. 6 determines an amount of the probe movement in direction and or angle based upon the electromagnetic radiation as reflected from the probe PB. Alternatively, the movement of the probe can be detected using a motion sensor, such as a MEMs sensor. Based upon the detected probe movement, the display icon of the current probe position i is also determined with respect to the patient body image PtBdy. Upon matching the position icons, additional visual feedback is optionally provided for matching the angle of the probe PB and the previously established angle among other things.

Without the above described visual aid, the operator relies only upon the anatomical landmarks of the scanned ultrasound image to identify the previously established position I. On the other hand, over the course of certain treatment, the landmarks may become unclear due to the visual changes in the region of interest. According to the exemplary process of the current invention, the previously established position I is ascertained based upon the above described visual aid that is based upon the probe PB position with respect to the patient PtBdy even without relying upon anatomical knowledge.

Based upon the probe tracking information as determined by the above described process, a set of previously stored images are selected from a storage device ST. The previously stored images includes the region of interest that has been currently scanned by the probe PB and are generally acquired by an imaging and diagnosis system of modalities such as Xray-based computer tomography (CT) and magnetic resonance imaging (MRI), which generally provides a higher resolution than the ultrasound imaging. A corresponding set of the high-resolution images is selected from the storage device ST for displaying based upon the probe tracking information.

Furthermore, the displayed data additionally includes other images that are generated from a variety of previously stored image data. For example, the displayed image is one of a 2D image, a 3D image and a 4D image that are based upon previously stored data and that corresponds to the change in spatial information with respect to the probe. Another exemplary displayed image is a 3D volume that is stitched together from a plurality of previously stored 3D volumes. Yet another exemplary displayed data is a 3D volume that is stitched together from a plurality of previously stored 2D images. An additional exemplary displayed image is an image that is based upon imaging data that is acquired by the probe that has been monitored for tracking.

FIG. 9 is a diagram illustrating a 3D image display as an exemplary application of the operator positional tracking in the image display system. For example, the tracking device 200 tracks the position of the head and or the eyes of the operator with respect to a predetermined reference or object such as a display monitor 120 within a predetermined space. As the operator moves his or her head from a first position A to a second position B, the position of the eyes are also changed with respect to the monitor 120. When the monitor 120 displays a 3D image, if the depth perception is achieved by a difference in the image in the right and left visual field of the operator, the monitor 120 has to update the image in the right and left visual field of the operator as the operator eye position changes. To accomplish this, the tracking device 200 tracks not only the operator whole body movement, but also the eye and or head position in order to properly maintain the depth perception. Although the above image display system is illustrated with respect to the ultrasound imaging and diagnostic systems, the above image display system is not limited to a particular imaging modality. Still referring to FIG. 9, the above described operator tracking optionally requires additional technology. One exemplary technology is facial recognition to accurately track the eye position of the operator. A facial recognition technology is also optionally combined to keep track of the identity of multiple operators. Theft of expensive imaging probes is a serious problem for medical facilities. The optical, IR camera and microphone could increase chance of the equipment recovery since it can record event when probe(s) are stolen. In order to protect patient and operator privacy, security monitoring should not be turned on all the time but it rather should be triggered by some event, e.g. probe removal etc. The position/location of the probe can be tracked using optical and magnetic techniques as is described above but may also be tracked using sensors embedded in the probe.

Some existing systems for 3D imaging use sophisticated mechanical devices to localize probe position and register 2D slice images in order to create 3D volumes. These devices are expensive, specialized for one specific type of exams, are bulky, and sometimes require a whole room for use.

The present embodiments address these issues and use free hand motions tracked by motion sensors, such as MEMs devices, as a replacement for complicated magneto-electrical-mechanical structures for creating 3D volumes. The use of free hand motions increases freedom and flexibility and overcomes the limitations of previously developed devices.

The motion sensor based devices of the present embodiment provide a better way of patient tracking for the purpose of improved imaging, image registration, and medical therapy.

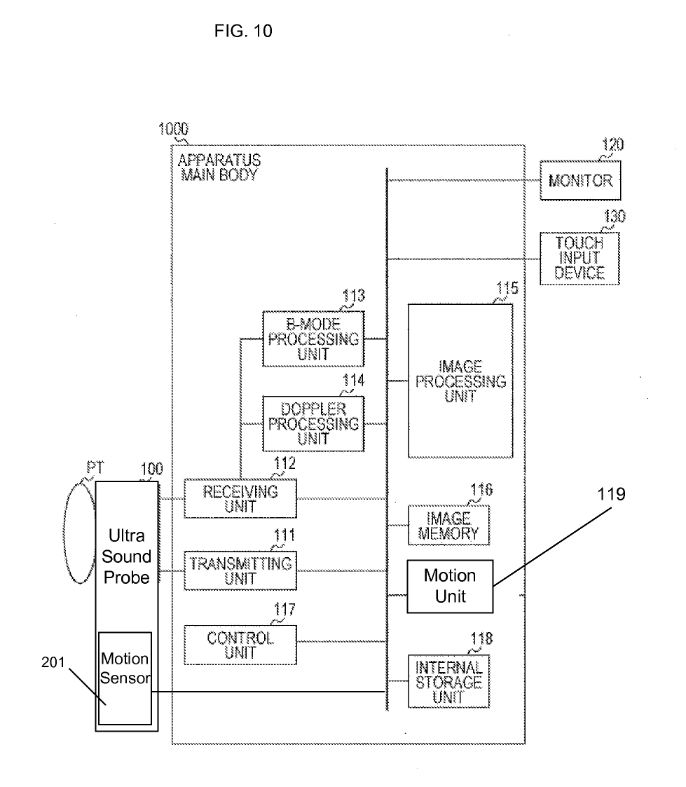

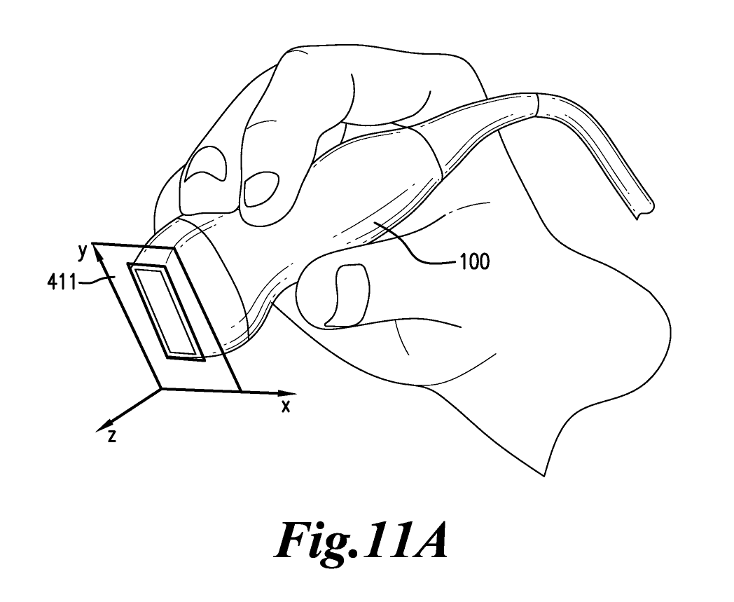

Now referring to FIG. 10, a diagram illustrates an exemplary configuration of one embodiment of the probe tracking device in the ultrasound imaging and diagnosis system. For example, in the embodiment, the tracking device 200 is replaced with a motion sensor 201 incorporated into or attached to the probe 100. The tracking technique in this embodiment is accomplished using a motion sensor 201 such as a 9-axis motion sensor for patient-probe-operator tracking. This motion sensor can be implemented as MEM device in silicon on insulator (“SOI”) technology.

The motion sensor provides detailed novel ways of probe tracking for the purpose of ultrasound image spatial registration and for the purpose of building extended 2D images, 3D volumes, extended 3D volumes, multimodality fusion, etc. The motion sensor also provides novel ways of patient tracking for the purpose improved imaging, image registration, and medical therapy. The elements found in FIG. 10 which are also shown in FIG. 1 have no been re-described in the present embodiment as the elements are equivalent unless otherwise noted. FIG. 10 further includes a movement calculation unit 119 implemented by processing circuitry. The movement calculation unit 119 described in further details below correlates the movement detected by the motion sensor with positions and locations or positions or locations in obtained imagery.

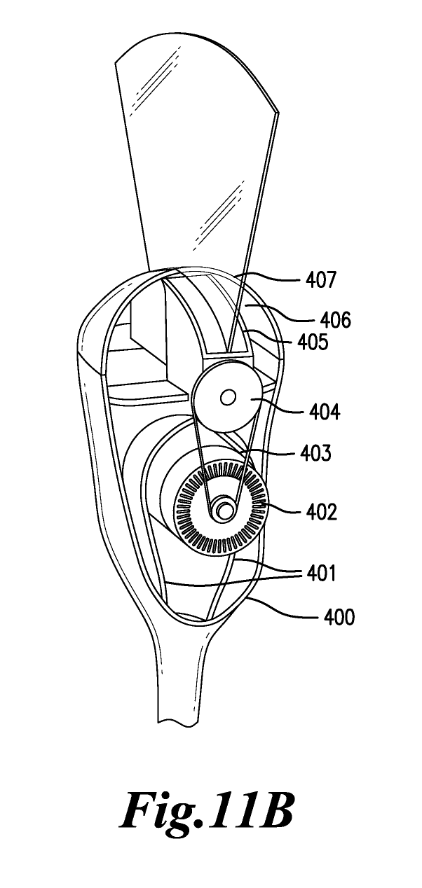

FIG. 11A illustrates an exemplary implementation of the motion sensor embodiment shown in FIG. 10. In this example, the probe 100 with motion sensor 201 embedded therein is utilized in place of an ultrasound wobbler.

A wobbler ultrasound probe is a 1D ultrasound array that is mechanically rotated along an elevation direction and is able to generate 3D ultrasound volumes. This 1D probe is disposed inside a housing 400 filled with ultrasound transparent fluid 406 as shown in FIG. 11B. The wobbler probe also includes cables 401, a position sensing device 402, a motor 403, a gear 404, and array 405, and an acoustic window 407. Wobbler ultrasound probes are expensive, heavy and hard to hold in the hand for prolonged periods often required by ultrasound imaging. However, they are nevertheless widely used as they provide good quality 3D imaging particularly in obstetric (“OB”) contexts.

The present implementation replaces the wobbler ultrasound probe with a standard 1D probe 100 having attached thereto a motion sensor 201, which when wobbled by hand along an elevation mimics the 3D wobbler probe functionality. The motion sensor 201 could be attached to probe 100 or probe handle or built into the probe 100 or probe handle. For instance, a 3-axis gyroscope motion sensor could track the probe 100 rotation to thereby enable proper 2D imaging slice registration and building of 3D volumes. In particular, the data obtained from the motion sensor 201 is transmitted to the movement calculation unit 119 which generates correction information which is used by the image processing unit 115 when registering 2D imaging slices and building the 3D volumes.

In general case, the motion sensor's Euclidean Space [U, V, W] is not aligned with 1D probe Euclidean space [X, Y, Z], where X is depth, Y is Lateral and Z is probe elevation as shown in FIG. 11A. During freehand wobbling, rotation is around the probe's Y axis but in motion sensor's space, rotation is arbitrary in a space defined by three axes [U, V, W] and can be described by a rotation matrix:

$$R = \vert \begin{matrix}{r}_{11} & {r}_{12} & {r}_{13} \\ {r}_{21} & {r}_{22} & {r}_{23} \\ {r}_{31} & {r}_{32} & {r}_{33}\end{matrix} \vert$$

According to Euler's rotation theorem, any rotation of rigid body about a fixed point is equivalent to a single rotation by angle θ about a fixed Euler axis defined by unit vector \vec{e}=(eₓ, e_{y}, e_{z}). Besides rotation matrix, there are other ways of tracking rotation but the simplest and most widely used is by use of quaternions. Quaternions give a simple way to encode any rotation of a rigid body by four numbers

q=(w, qₓ, q_{y}, q_{z}), where w=cos(θ/2), [qₓ, q_{y}, q_{z}]=[eₓ, e_{y}, e_{z}] sin(θ/2).

In cases when it is possible to align motion sensor Euclidean space with a 1D lateral probe, Euclidean space motion tracking will be significantly simplified during freehand wobbling.

The simplest approach is that wobbler rotation θ around a single axis is tracked and used for 3D image slice registration and rendering. Rotations around the other two axes and lateral motion along all three axes, are zeroed for the purpose of 3D registration.

It is difficult for humans to rotate around just a single probe axis Y during freehand wobbling. There are always some small rotations around other two axes which are taken into account in the more advanced approaches to 3D image registration. However 3D image quality is highest if the rotation around Y axis is dominant. In case that rotation around other two axes becomes larger than a predetermined value (e.g., 10% of rotation around Y axis), the user is warned that the created volume may be suboptimal.

During freehand wobbling, there should not be lateral probe sliding but human hands will nevertheless slide and this accidental sliding is tracked and taken into account during advanced image registration. Similarly to undesired rotation around two other axes, if lateral motion becomes larger than predetermined value (e.g., 10% of rotation around Y axis), the user is warned that the created volume may be suboptimal.

The highest image quality of freehand wobbling is achieved when rotation speed is uniform. However maintaining constant rotation speed is hard for humans so certain amount non-uniformity in rotation is tolerated, tracked and used in image registration. In addition if rotation speed variations are larger than predetermined threshold (e.g., 10%), the user is warned that the created volume is suboptimal.

These warnings about to large rotations, sidings and non-uniform rotation act as a training tool providing feedback to the user.

In past there have been a number of unsuccessful efforts to register images and create 3D volumes by use of image processing alone. The main problem was that cross correlation between successful images could not estimate the amount of movement along the line of probe motion. However 2D image correlation can be used as an additional tool in the correction of image registration along axes of unintentional motion. Thus, during probe wobbling around lateral axis, undesired sliding along depth and lateral axes can be corrected. The image correlation can be used together or in place of providing warnings to the user.

The 3D images subject to image registration based on the motion sensor 201 information is comparable in image quality to images obtained using 3D wobbler probes but at a fraction of the cost and with a much lower probe weight.

In order to avoid probe sliding during wobbling, a slide stopping device 411 could also be attached to probe nose as shown FIG. 11A.

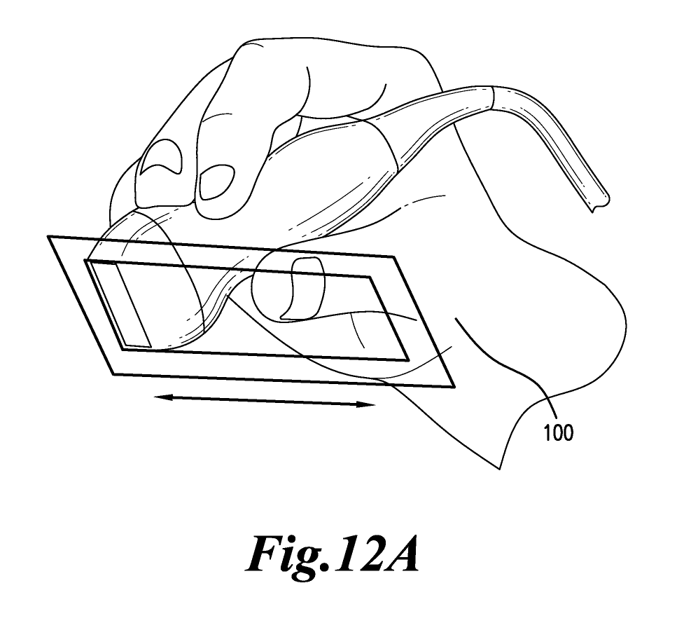

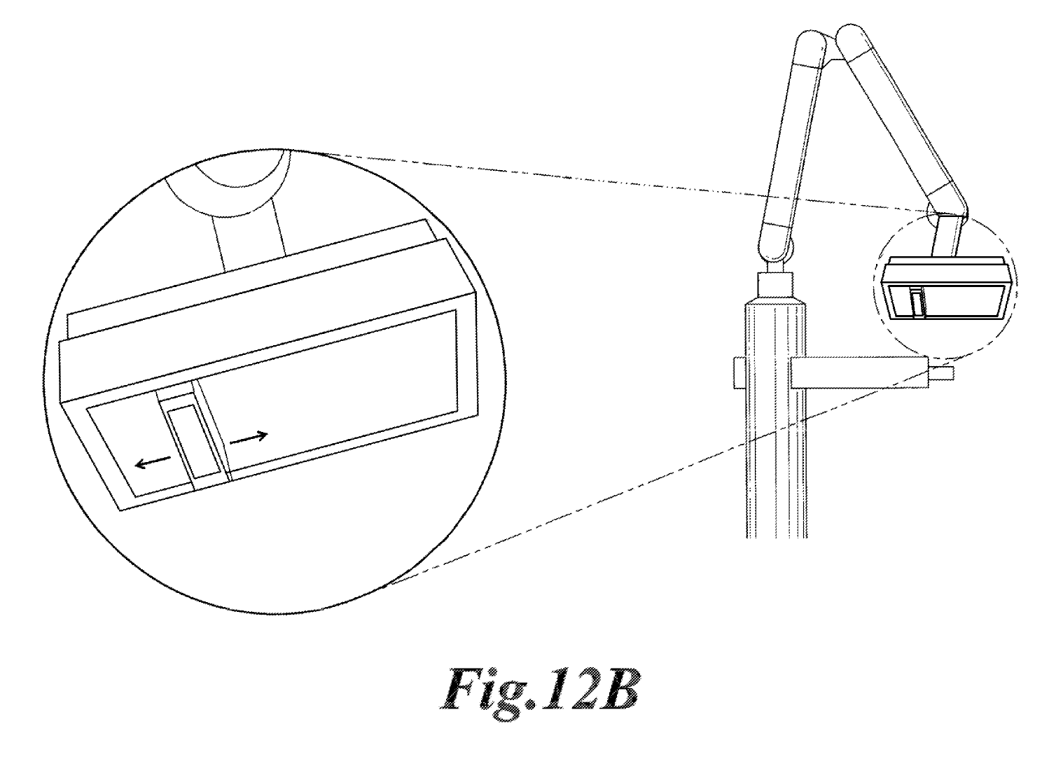

FIG. 12A illustrates a sliding probe exemplary implementation of the motion sensor embodiment shown in FIG. 10. There are various ultrasound imaging applications that could benefit from 3D images created by proper image registrations during probe sliding. One of the best is breast scanning. In this application, the probe 100 with motion sensor 201 is utilized in place of an Automated Breast Ultrasound Scanning (“ABUS”) system. As shown in FIG. 12B, in an ABUS system, a 1D probe linearly slides over special ultrasound transparent membrane while collecting 2D imaging slices that are combined into 3D volumes.

The present implementation replaces the ABUS system with a standard 1D probe 100 having attached thereto a motion sensor 201, which, when manually slid along elevation, mimics the functionality of the ABUS system. The motion sensor 201, which includes the 3-axis accelerometer and 3-axis gyroscope, provides tracking of the probe 100 motion and enables proper 2D imaging slice registration and building of 3D volumes. It is advantageous that sliding is linear. In particular, the data obtained from the motion sensor 201 is transmitted to the movement calculation unit 119, which generates correction information that is used by the image processing unit 115 when registering 2D imaging slices and building the 3D volumes.

Similarly to freehand wobbling, the simplest approach to image registration in the case of freehand sliding is to assume that motion is along a single straight line. This approach is even further simplified when the probe and motion sensor Euclidean spaces are aligned since the assumption will be that motion is along the elevation or the probe's Z axis.

Similarly to the case of wobbling, during freehand probe sliding the human hand will make undesired translation in directions other than the Z axis and will make undesired rotations. These rotations and slidings are taken into account in more advanced approaches to image rendering.

These undesired rotations and sidings can be monitored and a warning can be provided to the user leading toward the creation of more optimal 3D volumes. The warnings also provide a unique user training tool for more optimal image capturing operation.

During freehand 3D sliding the image processing based on image correlation is also able to provide additional correction of image rendering particularly in, but not limited to, the X and Y axis directions and for rotations around all three axes.

The 3D images subject to correction as a result of the motion sensor 201 information will be comparable in image quality to a 3D mechanical slider but will be provided but at a fraction of the cost and without complicated special equipment.

To make sliding more uniform, an ultrasound transparent membrane with gel on both sides may be placed on the patient's skin.

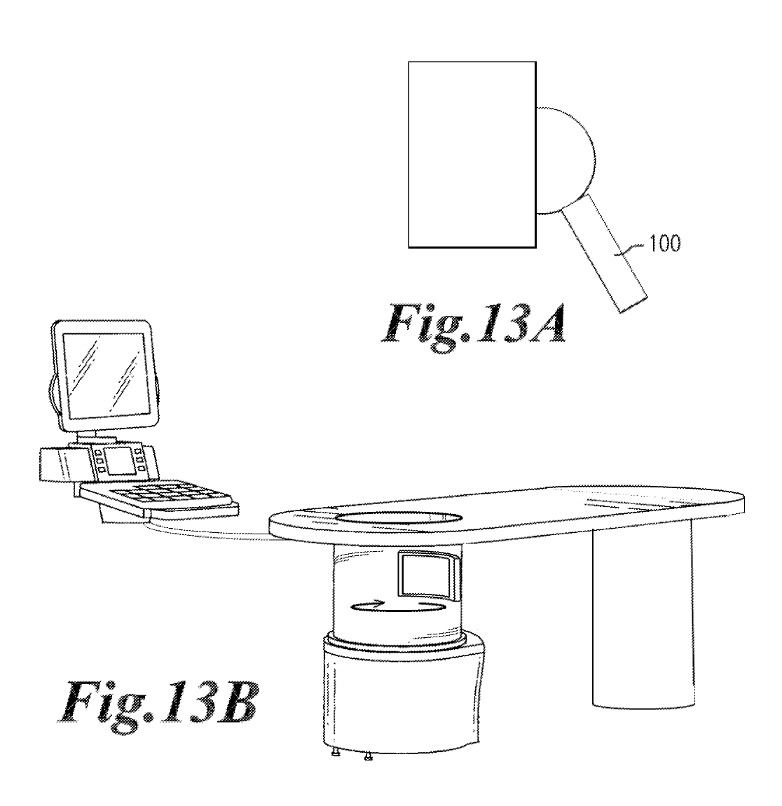

FIG. 13A illustrates an exemplary implementation of the motion sensor embodiment shown in FIG. 10. In this example, the probe 100 with motion sensor 201 is utilized together with ultrasound gel filled cups of ultrasound transparent material in shape of breast, or other body parts. This exemplary embodiment is used in place of SonoCine-AWBUS or ABUS-alternatives such as the Helix Medical System, Techniscan Medical System, etc., which scan the patient in a prone position or in a so called “Warm Bath Ultrasound system” as shown in FIG. 13B. These ABUS-alternatives were developed to address issues with the ABUS system. For instance, pressure created by the ABUS linear scan system alters breast anatomy making diagnostic correlation with other breast imaging modalities difficult. Furthermore, shearwave elastography technology cannot be used with ABUS systems. Further, ABUS images cannot be easily fused with mammography images.

The present embodiment is able to use ultrasound gel filled breast, or other body part, shaped cups of ultrasound transparent material. The ultrasound probe 100 with 6-axis or 9-axis motion sensor 201, such as a MEMs device, attached thereto is able to arbitrarily slide around the gel filled cups without changing breast shape. The 2D image slices are collected and registered for the purpose of creating 3D volumes by the image processing unit 115. The data obtained from the motion sensor 201 is transmitted to the movement calculation unit 119 which generates correction information which is used by the image processing unit 115 when registering 2D imaging slices and building the 3D volumes. The generated 3D volumes have similar characteristics to those generated by the ABUS-alternative systems and are comparable in image quality. Further, the present embodiment is able to provide these images at a fraction of the cost and complexity of the ABUS-alternative devices.

Thus the present alternative cup-device for ultrasound scanning can be created in order to bring breasts, or other body relevant body parts, into a good position for imaging such as a position used in mammography. Another advantage of this device is that compounded ultrasound 2D and 3D Images created using this alternative cup device would provide simple fusing with mammography images.

As noted, the present embodiment could be adopted for other body parts as well, for example for creating penile and testicle 3D volumes. During scanning other body parts, motion will be more complex with combination of rotations and linear sliding. These complex motions may be tracked and image registered for displaying 3D volumes. More advanced algorithms are able to track complex motion and help properly scan so that 3D volumes can be created.

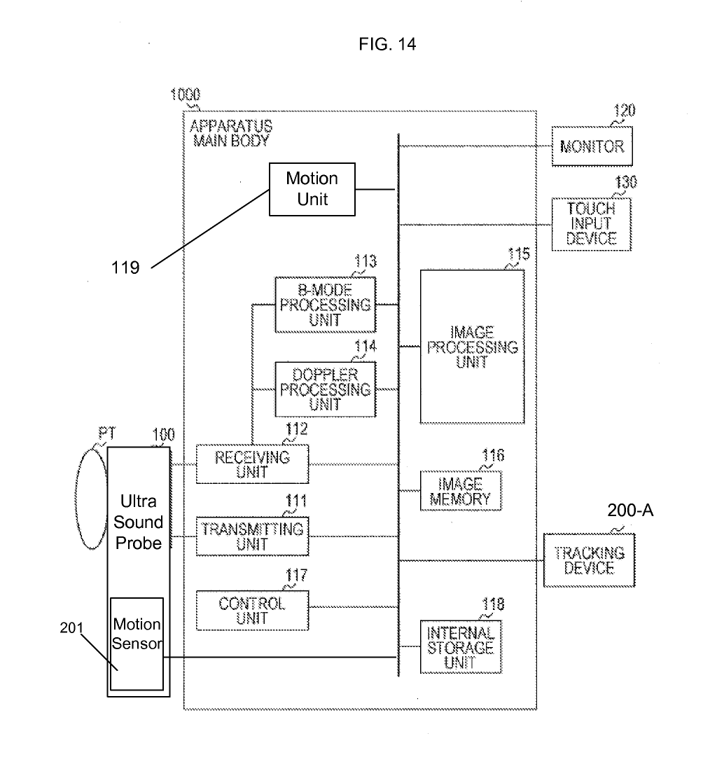

Now referring to FIG. 14, a diagram illustrates an exemplary configuration of one embodiment of the probe tracking device in the ultrasound imaging and diagnosis system. For example, in the embodiment the motion sensor 201 is incorporated into the probe 100, which is similarly illustrated in the embodiment described in FIG. 10. The elements found in FIG. 14 which are also shown in FIGS. 1 and 10, have not been re-described in the present embodiment as the elements are equivalent unless otherwise noted.

Motion sensors, such as MEMs devices, have good short term stability but often lack long term stability. In addition, motion sensors, such as MEMs, are able to track changes in position but do not provide an absolute location reference unless the initial position is established at the beginning by some setup procedure. However, an absolute location reference and motion sensor correction can be achieved by various techniques including 3D Optical Camera (e.g., Microsoft Xbox Kinect), ultrasound or other medical modality (MRI, CT, etc.), previously mentioned image correlation, magnetic sensors, etc.

In this embodiment, the tracking device 200-A can be any imaging system described above with reference to FIGS. 2A-C, for example. This implementation utilizes information from both the imaging tracking sensor 200-A and the motion sensor 201, where the motion sensor 201 can be any motion sensor described with reference to FIGS. 10-13A, for example. In one embodiment, the motion sensor 201 may be a MEMs sensor.

Motion sensing tracking may also be implemented by taking into account geometric imaging constraints. For example, during free hand sliding, the probe 100 surface could be maintained in a plane of 3D space as defined by the patient's skin surface.

In addition, a 3-axis gyroscope is often a more precise position locator than a 3-axis accelerometer and therefore the 3-axis gyroscope could be used as stabilizer for the accelerometer.

The system is able to provide the operator with free hand imaging guidance on the monitor 120. In particular, for any of the embodiments in the present application, guidance may be provided to the operator of the probe to guide the free hand imaging. For instance, the system could guide the operator by providing a visual or auditory indication which lets the operator know that the probe should be moved in a certain location, direction, speed, etc.

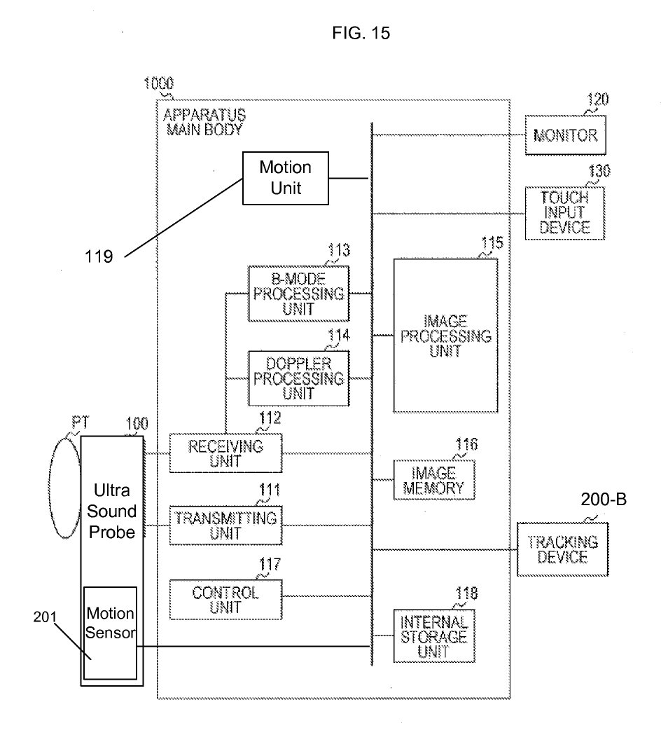

Now referring to FIG. 15, a diagram illustrates an exemplary configuration of one embodiment of the probe tracking device in the ultrasound imaging and diagnosis system. For example, in the embodiment, the motion sensor 201 is incorporated into the probe 100, which is similarly illustrated in the embodiment described in FIG. 10. The elements found in FIG. 14 which are also shown in FIGS. 1 and 10, have not been re-described in the present embodiment as the elements are equivalent unless otherwise noted. In this embodiment, an additional tracking device 200-B is used together with the motion sensor 201.

In each of FIGS. 10, 14 and 15, one or motion sensors 201 can be used and can be embedded in and connected to or embedding in or connected to the ultrasound probe. Each motion sensor 201 may be a motion sensor or a vibration sensor, or a position sensor, or a 3, 6 or 9 axis MEMs sensor or any combination thereof.

Multimodality image fusion with magnetic position sensors described above is achieved with initial sensor/probe setup procedure through multimodal image comparison. As is previously described with reference to FIG. 4, these magnetic sensors have a box that creates a magnetic field near the ultrasound probe 100. Magnetic coil(s) attached to the ultrasound probe 100 are used for probe localization and image registration. These coil(s) are connected to the main processing box by analog signals that run over multiple wires.

The present embodiment uses the 3-axis digital compass found in the motion sensor 201 instead of coils. The use of the motion sensor 201 simplifies integration into the probe 100 since the digital serial bus with as little as 2 wires used or no wires (if wireless connection used) is used for connection to the main body 1000. In addition, even existing ultrasound buses that typically run through probe connectors can be used for this purpose.

Another approach, when used in reference to FIG. 1, would be to use coils as disclosed above but to digitize the signals in the probe head or in the pod and to use the earlier mentioned digital bus that runs through probe connectors or use a wireless connection.

The motion sensor 201 could be connected to the system by hardwiring to control unit 117 and then to image processing unit 115 show in FIG. 10. The motion sensor 201 also can receive a power supply from the system along the similar path. The alternative option would be to have motion sensor signals bypass the control unit 117 and go directly to the image processing unit 115 via a proprietary bus or some standard bus, e.g. USB. In this case, the motion sensor's power supply could be directly provided from the image processing unit 115 as well.

Motion sensor utilization can be improved when the motion sensor 201 is wirelessly connected to the system. Bluetooth or similar standard communications protocols can be used for these purposes.

The motion sensor 201 can also be equipped with rechargeable batteries so that the motion sensor 201 can operate even if they are not directly connected to the system. This motion sensor 201 can utilize wireless charging using inductive chargers, e.g. Qi wireless charging protocols. This configuration simplifies device and system integration.

The wireless communication and charging capabilities produce relatively autonomous capabilities that enable additional advantages. For instance, the system having a motion sensor 201 that is charged and communicates or is charged or communicates wirelessly has the following advantages including: 1) locating remote controllers, bracelets, etc., that are easily misplaced; 2) tracking expensive medical equipment that can be damaged when dropped (e.g., ultrasound probes) or missing by theft; and 3) in the case of wireless or inductive charging, the lack of exposed charging connectors simplifies cleaning and sterilization and increases the probe's reliability since there are no connectors that are prone to failures.

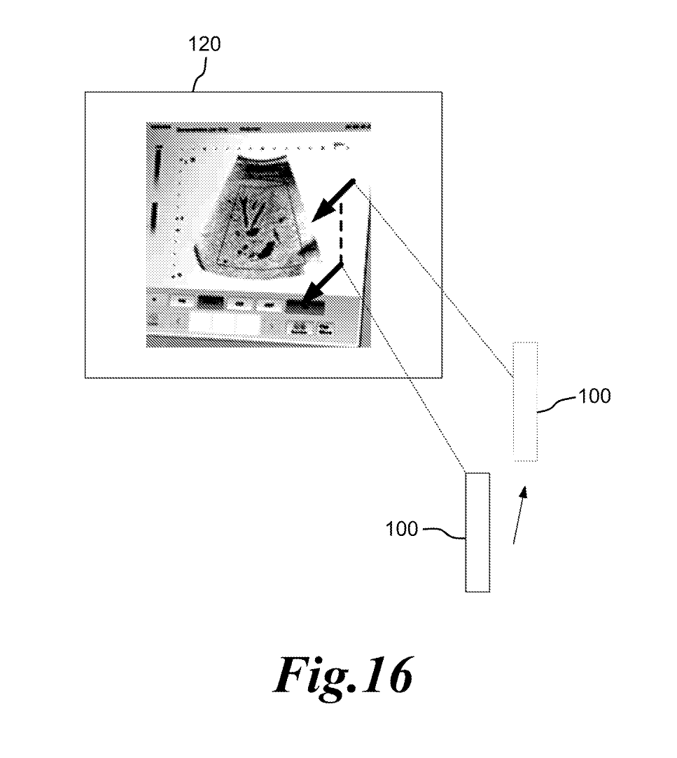

FIG. 16 illustrates an embodiment in which a motion sensor 201, such as the 9-axis MEMs device, is integrated into the ultrasound probe 100, to enable gesture control.

For instance, a special probe 100 with motion sensor 201 integrated therein can be used to control the ultrasound system. For instance, control can be implemented by “aiming” the probe 100 at on-screen controls, by shaking/tapping the probe 100 to initiate an action such as freeze/unfreeze, by tracking motions that the user creates, while the probe 100 is in hand, to thereby automate functions such as pictorial annotations and scan position annotations. The operation of the system based on detected movement of the probe 100 from the motion sensor 201 could be used in combination with audio/voice sensors and optical modeling/tracking or audio/voice sensors or optical modeling/tracking to expand commands and improve command accuracy.

For instance, a button or area on the screen could be selected in response to movement of the probe 100 having the motion sensor 201 including therein. Alternatively, the probe 100 movement could be followed by an on-screen cursor which is moved in response to the movement of the probe 100.

In addition, the probe 100 having the motion sensor 201 included therein can change imaging planes based on predetermined movements. This change of imaging planes could be implemented within a system user interface. The probe 100 having the motion sensor included therein can also be user to implement user interface (“UI”) commands. An example of such a command would be to interpret probe removal from a patient as a “freeze” command. Another example would be to interpret probe motion left-right along lateral axis as command for turning off/on BC mode and switching from/to B mode.

In an embodiment, the system includes a number of probes, similar to probe 100, which each is used for a different function of the system. For instance, one probe could be for adnominal, one for cardiology, one for prostate, one for nerve, one for obstetrics, one for gynecology, etc. Each probe 100 could include therein a motion sensor 201, such that when the probe 100 is moved by the operator, the system would enable this probe 100 (while keeping the other probes disabled or disabling the other probes) and would switch the system to operate for this particular probe. For instance, if a particular screen configuration is associated with one of the probes, this screen configuration could be displayed in response to the movement of the particular probe 100.

Motion sensors 201 may also be incorporated into other elements independent of the probe 100. For instance, a special remote control with motion sensor 201 integrated therein provides the user the ability to control an imaging system through “aiming” at onscreen controls, shaking/tapping the remote to initiate an action, track gestures the user creates while the control is in hand, or some combination of these inputs or other tracked movements of the remote control. As with the description above regarding the probe 100, the remote control could be used in combination with audio/voice sensors and optical modeling/tracking or audio/voice sensors or optical modeling/tracking to expand commands and improve command accuracy.

Motion sensors 201, such as MEMs sensors, may also be incorporated into wearable bands. The imaging system (CT, X-Ray, ultrasound, MR imaging) tracks gestures performed by the user, while the user wears a wrist mounted or hand mounted device or band including therein a motion sensor 201. The user is able to control the imaging system through “pointing” at on-screen controls and tracking gestures performed by the user while the control is in hand or some combination of these inputs. A specific advantage of a wearable solution would the ability to easily incorporate into the sterile environment. Such a wearable device could be used in combination with audio/voice sensors and optical modeling/tracking or audio/voice sensors or optical modeling/tracking to expand commands and improve command accuracy.

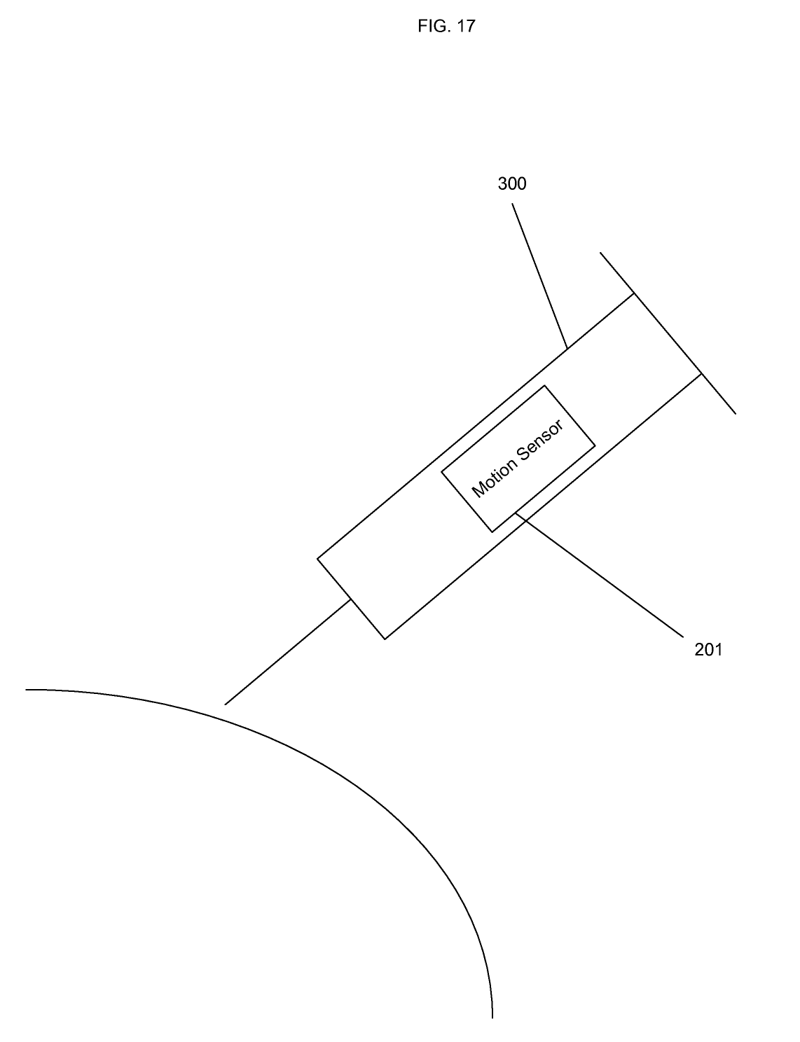

FIG. 17 illustrates an example of another embodiment in which one or more motion sensors 201 are incorporated into a biopsy needle 300. With regard to biopsy needles for tissue sampling, it is important to be able to precisely locate the tip of the needle during insertion in order to avoid sensitive organs (e.g., blood vessels, nerves, etc.) and to obtain a sample at a precise desired location. The present embodiment provides the important ability by integrating motion sensors 201, such as MEMs sensors, into the biopsy needle 300 in order to precisely track the needle. The motion sensor 201 could be a 6-axis inertial accelerometer/gyroscope or 9-axis MEMs based device, for example. The motion sensor 201 could also be implemented as a 3-axis digital magnetic compass. Alternatively, the motion sensor 201 could be replaced coils that are in the field created from a magnetic field box remote from the patient's skin.

The present embodiment has applications in X-Ray and computed tomography (“CT”) as well as ultrasound. The embodiment can be used in magnetic resonating imaging if the probe and motion sensor 201 are nonferrous. In CT, depending on the size and composition of the motion sensor 201, a metal artifact reduction algorithm can also be applied to improve accuracy.

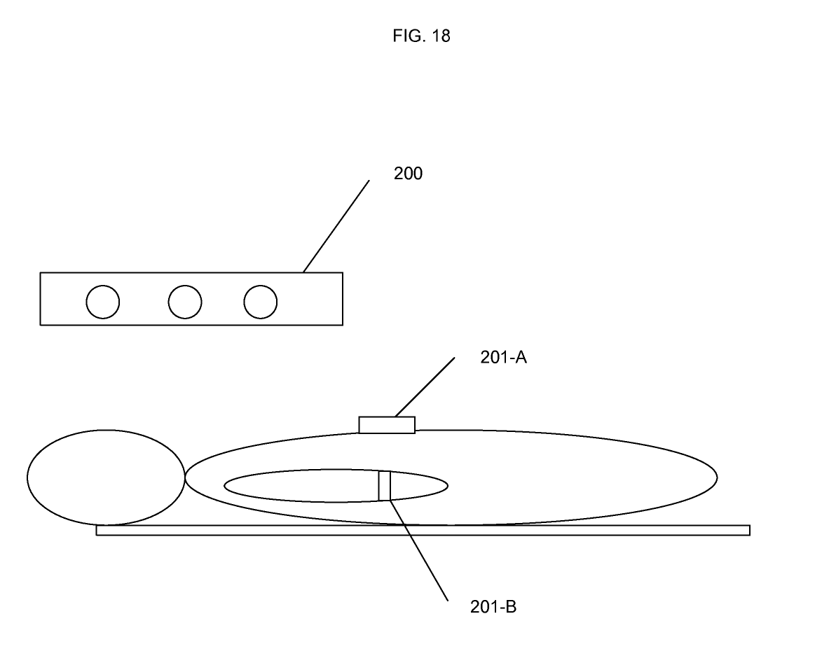

FIG. 18 illustrates an example of another embodiment in which one or more motion sensors 201 (201-A and 201-B) are associated with a patient for breathing and/or patient motion tracking. For instance, in this embodiment, motion sensors 201 can be placed on the skin of the patient 201-A, within the patient, or on a device that is worn by the patient 201-B or some combination thereof. Data from a wearable motion sensor 201-A or 201-B could be integrated with acquired imaging data to track, anticipate and correct for breathing and patient motion within the images. For instance, the information for the one or more motion sensors 201 could be transmitted to the motion processing unit 119 shown in FIG. 10 and used to generate correction data which is applied to the image processing unit 115 which performs registration of obtained images based on the correction data.

When acquisition of volume image data is subject to distortion from typical patient physiologic motions like breathing, tracking the motion associated with breathing is utilized to correct for motion or gate/remove/ignore data that is gathered during the displacement portions of the breathing cycle.

During ultrasound therapy it is often important to monitor the amount of delivered energy in order to treat disease but avoid excessive energy that could cause damage to healthy surrounding organs or tissue. Human motion that cannot be avoided poses serious problems with regard to controlling therapy dose. By tracking motion and the position of human organs via motion sensors 201, such as MEMs based sensors, or by imaging based tracking or some combination thereof, is able to significantly increase precision in therapy dose accuracy.

For instance, in diagnostic imaging, the dose is often determined based on an unrealistic scenario of prolonged imaging at a single location. By precisely tracking dose delivery at various locations during diagnostic imaging, the system is able to deliver more precise and sometimes higher values of power at each of the various locations.

Correcting for breathing motion is also applicable in several clinical applications for CT, X-ray and MR imaging in addition to uses in ultrasound described above.

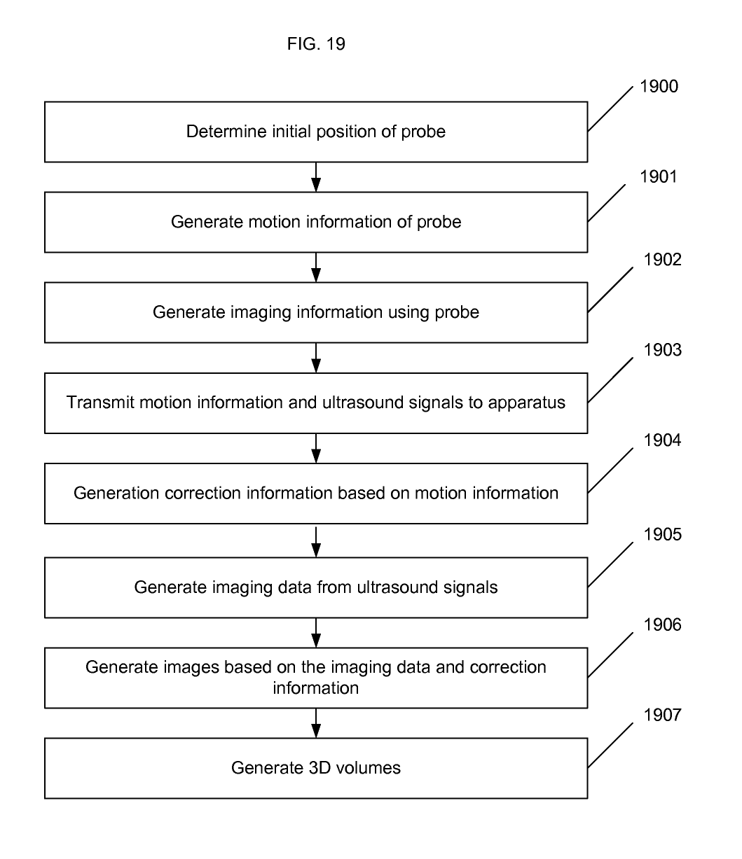

FIG. 19 describes a process for tracking motion in a probe 100 or any other element. In step 1900, information about the initial position of the probe is determined. This initial position can be obtained based on using optical or magnetic sensors or via a predetermined position value (such as in position in a holder). In step 1901, information about the motion of the probe 100 is generated from the motion sensor 201. In step 1902, ultrasound signals are obtained using the probe 100 concurrently with the generation of the motion information. In step 1903, the motion information and the ultrasound signals are transmitted to the apparatus main body 1000. Specifically, the ultrasound signals are transmitted to the receiving unit 112 and the motion information is transmitted to the motion processing unit 119. In step 1904, the motion processing unit 119 generates correction information based on the motion information. In step 1905, the Doppler processing unit 114 or the b-mode processing unit 113 generate information from the ultrasound signals. In step 1906, the image processing unit 115 generates ultrasound display images such as B-mode images or Doppler images for a display device taking into account the corresponding correction information generated by the motion processing unit 119. In step 1907, 3D volume is rendered.

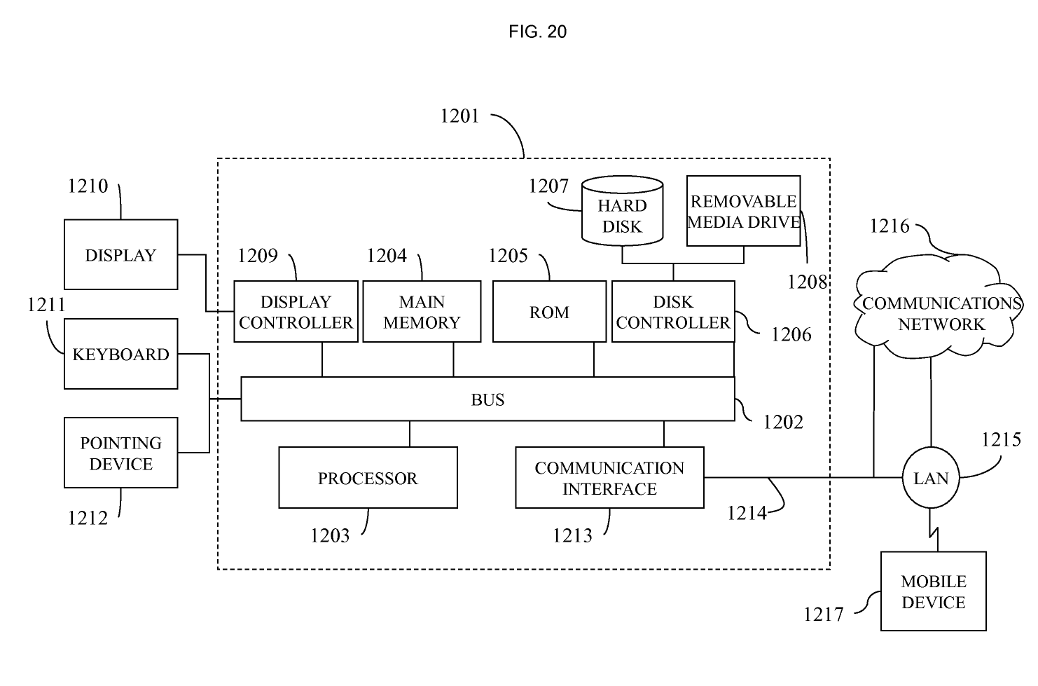

The processing units, such as but not limited to the B-mode processing unit 113, the Doppler processing unit 114, the image processing unit 115, the control unit 117, the motion processing unit 119, etc. described above with reference to FIGS. 1, 10, 14 and 15 can be implemented using a computer system or programmable logic. FIG. 20 illustrates a computer system 1201 upon which embodiments of the present disclosure may be implemented. The computer system 1201 may include the various above-discussed components with reference: to FIGS. 3-5, which perform the above-described process.

The computer system 1201 includes a disk controller 1206 coupled to the bus 120210 control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 (or display adapter 340) coupled to the bus 1202 to control a display 1210 (or display 360) such as a liquid crystal display (LCD), for displaying information to a computer user. The computer system includes input devices, such as a keyboard 1211 and a pointing device 1212, for interacting with a computer user and providing information to the processor 1203 (or processing device/unit 320). The pointing device 1212, for example, may be a mouse, a trackball, a finger for a touch screen sensor, or a pointing stick for communicating direction information and command selections to the processor 1203 and for controlling cursor movement on the display 1210.

The computer system 1201 performs a portion or all of the processing steps of the present disclosure in response to the processor 1203 executing one or more sequences of one or more instructions contained in a memory, such as the main memory 1204 (or memory 330). Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the present disclosure and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes.

Stored on any one or on a combination of computer readable media, the present disclosure includes software for controlling the computer system 1201, for driving a device or devices for implementing the invention, and for enabling the computer system 1201 to interact with a human user. Such software may include, but is not limited to, device drivers, operating systems, and applications software. Such computer readable media further includes the computer program product of the present disclosure for performing all or a portion (if processing is distributed) of the processing performed in implementing the invention.

The computer code devices of the present embodiments may be any interpretable or executable code mechanism, including but not limited to scripts, interpretable programs, dynamic link libraries (DLLs), Java classes, and complete executable programs. Moreover, parts of the processing of the present embodiments may be distributed for better performance, reliability, and/or cost.

The term “computer readable medium” as used herein refers to any non-transitory medium that participates in providing instructions to the processor 1203 for execution. A computer readable medium may take many forms, including but not limited to, non-volatile media or volatile media. Non-volatile media includes, for example, optical, magnetic disks, and magneto-optical disks, such as the hard disk 1207 or the removable media drive 1208. Volatile media includes dynamic memory, such as the main memory 1204. Transmission media, on the contrary, includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be carried on a magnetic disk of a remote computer. The remote computer can load the instructions for implementing all or a portion of the present disclosure remotely into a dynamic memory and send the instructions over a telephone line using a modem. A modem local to the computer system 1201 may receive the data on the telephone line and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an integrated services digital network (ISDN) card. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216 use, for example, electrical, electromagnetic, or optical signals that carry digital data streams, and the associated physical layer (e.g., CAT 5 cable, coaxial cable, optical fiber, etc.). The signals through the various networks and the signals on the network link 1214 and through the communication interface 1213, which carry the digital data to and from the computer system 1201 may be implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214 and the communication interface 1213. Moreover, the network link 1214 may provide a connection through a LAN 1215 to a mobile device 1217 such as a personal digital assistant (PDA) laptop computer, or cellular telephone.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

Furthermore, the above embodiments are described with respect to examples such as devices, apparatus and methods. Another embodiment to practice the current invention includes computer software such as programs for tracking a predetermined combination of an ultrasound probe, an operator and a patient for the ultrasound system that is loaded into a computer form a recording medium where it is stored.

It is noted that, as used in the specification, the singular forms “a,” “an,” and “the” may also include plural referents unless the context clearly dictates otherwise.

1. An ultrasound probe, comprising:
a housing;
a transducer located inside said housing having transducer elements in a predetermined configuration that generates ultrasound, transmits the generated ultrasound towards an object, and receives echoes that have been reflected from the object;
a motion sensing device having a fixed position with respect to said housing and including a sensor configured to generate a movement signal indicative of at least one type of movement of the ultrasound probe; and
processing circuitry configured to generate images from the echoes received by the transducer and to correct the generated images by image correlation based on the generated movement signal.

2. The ultrasound probe according to claim 1, wherein said motion sensing device is located inside said housing.

3. The ultrasound probe according to claim 1, wherein said motion sensing device is located outside said housing.

4. The ultrasound probe according to claim 1, further comprising a mounting frame for detachably mounting said motion sensing device on said housing.

5. The ultrasound probe according to claim 1, wherein said motion sensing device includes any combination of a gyroscope, an accelerometer, and a compass.

6. The ultrasound probe according to claim 1, wherein said motion sensing device includes at least a microelectromechanical (MEM) device.

7. The ultrasound probe according to claim 1, wherein said motion sensing device generates the movement signal indicative any combination of a linear movement, a rotational movement, an angular movement, and acceleration.

8. The ultrasound probe according to claim 1, further comprising a controller connected to said motion sensing device configured to selectively activate said motion sensing device.

9. The ultrasound probe according to claim 1, wherein the processing circuitry is further configured to provide a warning to the user of the ultrasound probe when the movement signal indicates movement having a value greater than a predetermined threshold.

10. A method of obtaining motion related data for an ultrasound probe, comprising:
providing a motion sensing device at a fixed position with respect to a housing of the ultrasound probe;
generating a movement signal indicative of at least one type of movement of the ultrasound probe using a sensor of the motion sensing device;
receiving echoes from an object that has been transmitted with ultrasound;
generating images from the echoes received by the transducer; and
correcting the generated images by image correlation based on the generated movement signal.

11. The method of obtaining motion related data for an ultrasound probe according to claim 10, wherein the correcting further includes correlating images by compensating for linear movement in non-designated directions and rotational movement in non-designated axes.

12. The method of obtaining motion related data for an ultrasound probe according to claim 10, wherein said providing step places the motion sensing device inside said housing.

13. The method of obtaining motion related data for an ultrasound probe according to claim 10, wherein said providing step places the motion sensing device outside said housing.

14. The method of obtaining motion related data for an ultrasound probe according to claim 10, further comprising an additional step of detachably mounting the motion sensing device on the housing.

15. The method of obtaining motion related data for an ultrasound probe according to claim 10, wherein the signal is generated using any combination of a gyroscope, an accelerometer, and a compass.

16. The method of obtaining motion related data for an ultrasound probe according to claim 10, wherein said providing step uses at least a microelectromechanical (MEM) device.

17. The method of obtaining motion related data for an ultrasound probe according to claim 10, wherein said generating step generates the signal indicative any combination of a linear movement, a rotational movement, an angular movement, and acceleration.

18. The method of obtaining motion related data for an ultrasound probe according to claim 10, further comprising an additional step of selectively activating the motion sensing device.

19. The method of obtaining motion related data for an ultrasound probe according to claim 10, wherein the signal is generated while the probe is reciprocated over a surface of a patient in a substantially linear manner.

20. The method of obtaining motion related data for an ultrasound probe according to claim 10, wherein the signal is generated while the probe is repeatedly wobbled at a pivoted area of a patient.

21. The method of obtaining motion related data for an ultrasound probe according to claim 10, wherein the signal is generated as the probe is moved in a predetermined unique manner to indicate a predefined meaning during an ultrasound examination.

22. The method of obtaining motion related data for an ultrasound probe according to claim 21, wherein the predefined meaning is a beginning of data collection.

23. The method of obtaining motion related data for an ultrasound probe according to claim 21, wherein the predefined meaning is an ending of data collection.

24. The method of obtaining motion related data for an ultrasound probe according to claim 10, further comprising:
providing a warning to the user of the ultrasound probe when the movement signal indicates movement having a value greater than a predetermined threshold.
